(19) United States
(12) Patent Application Publication
(10) Pub. No.: US 2024/0350262 A1
(43) Pub. Date: Oct. 24, 2024

(54) MECHANICALLY-EXPANDABLE PROSTHETIC VALVE

(71) Applicant: EDWARDS LIFESCIENCES CORPORATION, Irvine, CA (US)

(72) Inventors: Noam Miller, Givatayim (IL); Anatoly Dvorsky, Haifa (IL); Eran Grosu, Kfar-Saba (IL); Michael Bukin, Pardes Hanna (IL); Nikolai Gurovich, Hadera (IL)

(21) Appl. No.: 18/761,769

(22) Filed: Jul. 2, 2024

Related U.S. Application Data
(63) Continuation of application No. PCT/US2023/010622, filed on Jan. 11, 2023.
(60) Provisional application No. 63/409,343, filed on Sep. 23, 2022.
(60) Provisional application No. 63/404,675, filed on Sep. 8, 2022.
(60) Provisional application No. 63/298,526, filed on Jan. 11, 2022.

(51) Int. Cl. A61F 2/24

(52) U.S. Cl. CPC A61F 2/2418; A61F 2/243

(57) ABSTRACT

A prosthetic valve includes a frame having a first frame portion and a first window formed in the first frame portion. A threaded rod is coupled to the first frame portion and extends through the first window. Rotation of the threaded rod relative to the first frame portion radially expands or radially compresses the frame. A stopper is disposed within the first window and coupled to the threaded rod. The stopper is fixed in axial position relative to the threaded rod and axially movable within the first window by rotation of the threaded rod. The stopper can engage a first edge of the window to produce radial compression of the frame.

### CROSS-REFERENCE TO RELATED APPLICATIONS

This application is a continuation of PCT Application No. PCT/US2023/10622, filed on Jan. 11, 2023, which claims the benefit of U.S. Provisional Patent Application No. 63/404,675, filed on Sep. 8, 2022, U.S. Provisional Patent Application No. 63/298,526, filed on Jan. 11, 2022, and U.S. Provisional Patent Application No. 63/409,343, filed on Sep. 23, 2022, each of which is incorporated by reference herein in its entirety.

### FIELD

The present disclosure relates to implantable, mechanically expandable prosthetic heart valves and frame structures for use with mechanically expandable prosthetic heart valves.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable.

In one specific example, a prosthetic valve can be mounted in a crimped state on the distal end of a delivery device and advanced through the patient's vasculature (for example, through a femoral artery and the aorta) until the prosthetic valve reaches the implantation site in the heart. The prosthetic valve is then expanded to its functional size, for example, by inflating a balloon on which the prosthetic valve is mounted, actuating a mechanical actuator that applies an expansion force to the prosthetic valve, or by deploying the prosthetic valve from a sheath of the delivery device so that the prosthetic valve can self-expand to its functional size.

Prosthetic valves that rely on a mechanical actuator for expansion can be referred to as “mechanically-expandable” prosthetic valves. Mechanically-expandable prosthetic valves can provide one or more advantages over self-expandable and balloon-expandable prosthetic valves. For example, mechanically-expandable prosthetic valves can be expanded to various diameters. Mechanically-expandable prosthetic valves can also be compressed after an initial expansion (for example, for repositioning and/or retrieval).

Mechanically-expandable prosthetic valves can include frames that can be radially compressed and/or expanded by means of a mechanical actuator. Despite the recent advances in mechanically-expandable prosthetic valves, there remains a need for improved frames for mechanically-expandable prosthetic valves.

### SUMMARY

Disclosed herein are examples of a prosthetic valve that can be delivered to an implantation location within a patient's body and examples of a method of delivering the prosthetic valve.

In some examples, a prosthetic valve includes a frame having an axial direction extending from an inflow end to an outflow end, a first frame portion extending in the axial direction, and a first window formed in the first frame portion. The prosthetic valve includes a threaded rod coupled to the first frame portion and extending through the first window. Rotation of the threaded rod relative to the first frame portion in a first direction radially expands the frame, and rotation of the threaded rod relative to the first frame portion in a second direction that is opposite to the first direction radially compresses the frame. The prosthetic valve includes a stopper disposed within the first window and coupled to the threaded rod. The stopper is fixed in axial position relative to the threaded rod and axially movable within the first window by rotation of the threaded rod. The stopper can engage a first edge of the window to produce radial compression of the frame.

In some examples, a prosthetic valve includes a frame having an axial direction extending from an inflow end to an outflow end, a first frame portion extending in the axial direction, a second frame portion extending in the axial direction, a first window formed in the first frame portion, a second window formed in the second frame portion. The first window is positioned axially between the second window and the outflow end in the axial direction. The prosthetic valve includes a threaded rod coupled to the first frame portion and extending through the first window. Rotation of the threaded rod relative to the first frame portion in a first direction radially expands the frame, and rotation of the threaded rod relative to the first frame portion in a second direction that is opposite to the first direction radially compresses the frame. The prosthetic valve includes a stopper fixedly coupled to the threaded rod and disposed within the first window. The stopper can engage a first edge of the first window to produce radial compression of the frame. A valvular structure is disposed inside the frame and has a commissure received in the second window.

In some examples, a prosthetic valve includes a frame having an axial direction extending from an inflow end to an outflow end, a first frame portion extending in the axial direction, the first frame portion comprising a first actuator portion connected to the inflow end at a first apex, a second actuator portion connected to the outflow end at a second apex, and a recess formed in the second apex. The prosthetic valve includes a threaded rod having a head portion. The threaded rod extends through the first actuator portion and the second actuator portion and is threadedly engaged with the first actuator portion with the head portion received in the recess. A height of the head portion is equal to or less than a depth of the recess. Rotation of the threaded rod relative to the first actuator portion in a first direction radially expands the frame, and rotation of the threaded rod relative to the first actuator portion in a second direction that is opposite to the first direction radially compresses the frame.

In some examples, a prosthetic valve includes a frame having an axial direction extending from an inflow end to an outflow end, a plurality of first frame portions spaced about a circumference of the frame, and a first window formed in each of the first frame portions. The prosthetic valve includes a plurality of threaded rods. Each threaded rod is coupled to one of the first frame portions and extends through the respective first window formed in the one of the first frame portions. Rotation of each threaded rod relative to the respective first frame portion in a first direction radially expands the frame, and rotation of each threaded rod relative to the respective first frame portion in a second direction that is opposite to the first direction radially compresses the frame. The prosthetic valve includes at least one stopper fixedly coupled to one of the threaded rods and disposed within the first window receiving the one of the threaded rods. The at least one stopper can engage a first edge of the respective first window to produce radial compression of the frame.

In some examples, a method includes coupling a prosthetic valve to a distal end of a delivery apparatus. The prosthetic valve includes a frame having an axial direction extending from an inflow end to an outflow end and having a first actuator portion and a second actuator portion aligned along the axial direction, a threaded rod coupled to the first actuator portion and extending axially through the second actuator portion, and a stopper fixedly coupled to the threaded rod and disposed within a window formed in the second actuator portion. The method includes rotating the threaded rod in a first direction to cause the stopper to engage a first edge of the window, thereby causing the first actuator portion to move away from the second actuator portion and radially compress the prosthetic valve.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the invention will become more apparent from the following detailed description, which proceeds with reference to the accompanying figures.

### BRIEF DESCRIPTION OF THE DRAWINGS

FIG. 1 depicts a perspective view of a prosthetic heart valve according to one example, shown in a radially expanded configuration.

FIG. 2 depicts a side view of a portion of the frame of the prosthetic heart valve of FIG. 1.

FIG. 3 depicts a side view of the frame of the prosthetic heart valve of FIG. 1, shown in a radially contracted configuration with no barreling of the frame.

FIG. 4 depicts a side view of the frame of the prosthetic heart valve of FIG. 1, shown in a radially contracted configuration with barreling of the frame.

FIG. 5 depicts a side view an exemplary delivery assembly comprising the prosthetic heart valve of FIG. 1 and an exemplary delivery apparatus, which can be used with any of the prosthetic heart valves disclosed herein.

FIG. 6A depicts a side view of a distal end portion of the delivery assembly of FIG. 5, showing the frame of the prosthetic heart valve deployed from within a delivery capsule of delivery apparatus and in a radially expanded configuration.

FIG. 6B depicts a side view of the distal end portion of the delivery assembly of FIG. 5, showing the frame of the prosthetic heart valve in a radially compressed configuration.

FIG. 6C depicts a side view of the distal end portion of the delivery assembly of FIG. 5, showing a delivery configuration in which the prosthetic heart valve is disposed within the delivery capsule of the delivery apparatus in a radially compressed configuration.

FIG. 7 depicts a side view of a portion of the delivery assembly, showing the prosthetic heart valve retained in the radially compressed configuration by an adjustable loop of the delivery apparatus.

FIG. 8 depicts a side view of a section of a frame for a prosthetic heart valve according to one example, the frame comprising apertures in one vertical strut.

FIG. 9A depicts a side view of a section of a frame for a prosthetic heart valve according to some example.

FIG. 9B depicts a side view of a section of a frame for a prosthetic heart valve according to some examples, the frame comprising a commissure opening.

FIG. 10A depicts an end view of a section of a frame for a prosthetic heart valve, the frame comprising vertical struts projecting radially inwards.

FIG. 10B depicts an end view of a frame section for a prosthetic heart valve, the frame comprising a commissure opening projecting radially inwards.

FIG. 11 depicts a side view of a frame section comprising lateral support members according to one example.

FIG. 12 depicts a side view of a frame section comprising lateral support members according to some example.

FIG. 13 depicts a side view of a section of a frame for a prosthetic heart valve comprising a collapsible aperture according to one example.

FIG. 14 depicts a radially interior side view of a section of the frame shown in FIG. 13 operatively coupled to an actuation assembly and in a radially compressed configuration.

FIG. 15 depicts a radially interior side view of a section of the frame shown in FIG. 13 operatively coupled to an actuation assembly and in a radially expanded configuration.

FIG. 16A is a perspective view of a vertical post with a collapsible aperture according to one example.

FIG. 16B is a side elevation view of the vertical post shown in FIG. 16A in the axially extended state.

FIG. 16C is a side elevation view of the vertical post shown in FIG. 16A in the axially compressed state.

FIG. 17 is a graphical depiction of the correlation between the forces acting on a vertical post according to one example, and the resulting compressive deformation of the vertical post.

FIG. 18A is a perspective view of one example of a prosthetic valve including a frame and a plurality of leaflets attached to the frame.

FIG. 18B is a perspective view of the prosthetic valve of FIG. 18A with an outer skirt disposed around the frame.

FIG. 19A is a perspective view of a frame for the prosthetic valve of FIG. 18A.

FIG. 19B is a front portion of the frame shown in FIG. 19A.

FIG. 20A is an enlarged view of a frame portion of FIG. 19B showing a rod actuator coupled to the frame.

FIG. 20B is the frame portion of FIG. 20A showing a head portion of a rod actuator protruding from an outflow end of the frame.

FIG. 21 is a side elevation view of a delivery apparatus for a prosthetic device, such as a prosthetic valve, according to one example.

FIG. 22A is a perspective view of a portion of a threaded rod engaged with an actuator assembly of a delivery apparatus, according to one example.

FIG. 22B is a perspective view of the threaded rod of FIG. 22A disengaged from the actuator assembly.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present, or problems be solved.

Although the operations of some of the disclosed examples are described in a particular, sequential order for convenient presentation, it should be understood that this manner of description encompasses rearrangement, unless a particular ordering is required by specific language set forth herein. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (for example, out of the patient's body), while distal motion of the device is motion of the device away from the user and toward the implantation site (for example, into the patient's body). The terms “longitudinal” and “axial” refer to an axis extending in the proximal and distal directions, unless otherwise expressly defined.

As used herein, “e.g.” means “for example,” and “i.e.” means “that is.”

As used herein, the term “parallel” refers to an orientation between a component and a reference line that is parallel or substantially parallel, allowing for minor angular orientation or curvature. When discussing the orientation of a component with an elongated geometry, such as a strut or actuator, the orientation of that component is defined by an axis drawn along the component's length (that is, a major axis of the component), through the cross-sectional midpoint of the component. When the component being discussed has a bend or curvature, the axis is drawn through the cross-sectional midpoint of each endpoint of the component along the length of the component. For example, the axis can, in the case of a curved component, be defined by a chord extending between the ends of the component and drawn through the cross-sectional midpoint at each end.

The axis of a component can be substantially parallel to a reference line if only a small angle, such as 10 degrees or less, exists between the component and the reference line. Thus, for example, an axis of a component may be described as extending parallel to a reference line (such as a vertical axis of a frame) if it is parallel to the reference line, or within 10 degrees of parallel to the reference line.

### Introduction to the Disclosed Technology

Disclosed herein are various examples of prosthetic heart valves for implantation in the native vasculature of a patient, such as the native annuluses of the patient's heart (for example, the aortic, pulmonary, mitral, or tricuspid valves). The disclosed prosthetic heart valves can also be implanted within vessels in communication with the heart, including a pulmonary artery (for replacing the function of a diseased pulmonary valve, or the superior vena cava or the inferior vena cava (for replacing the function of a diseased tricuspid valve) or various other veins, arteries, and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For instance, in one example, the disclosed prosthetic valves can be implanted within a docking device implanted within the pulmonary artery for replacing the function of a diseased pulmonary valve, such as disclosed in U.S. Patent Publication No. 2017/0231756, which is incorporated by reference herein. In some examples, the disclosed prosthetic valves can be implanted within a docking device implanted within or at the native mitral valve, such as disclosed in PCT Publication No. WO2020/247907, which is incorporated herein by reference. In some examples, the disclosed prosthetic valves can be implanted within a docking device implanted within the superior or inferior vena cava for replacing the function of a diseased tricuspid valve, such as disclosed in U.S. Patent Publication No. 2019/0000615, which is incorporated herein by reference.

To facilitate implantation within a patient, prosthetic valves disclosed herein can be radially compressible and expandable between a radially compressed state and a radially expanded state. Thus, the prosthetic valves can be crimped on or retained by an implant delivery apparatus in the radially compressed state during delivery, such as by a delivery system catheter or adjustable loop disposed around the compressed prosthetic heart valve. The prosthetic heart valves can then be expanded by an expansion mechanism, such as an actuator, to the radially expanded state once the prosthetic valve reaches the implantation site. The frames can also be locked in the desired state of radial expansion by means of a locking mechanism, thereby preventing further radial expansion or compression of the prosthetic heart valve frame. It is understood that the prosthetic valves disclosed herein may be used with a variety of implant delivery apparatuses and can be implanted via various delivery procedures, examples of which is discussed herein.

Because reduced prosthetic heart valve diameters are generally preferred for ease of implantation, and because greater radial compression results in greater axial extension while the prosthetic heart valve is in the radially compressed configuration, it can be advantageous to design the prosthetic heart valve with longer, thinner actuators that support a greater range of axial extension while minimizing the radial profile of the compressed prosthetic heart valve.

The radial compression of the prosthetic heart valves, however, can pose several technical challenges. In particular, the prosthetic heart valve may not radially compress evenly along its axial length, instead radially compressing to a greater degree at either axial end, and to a lesser degree towards the axial center of the prosthetic heart valve. This can result in the prosthetic heart valve frame assuming a “barreled” shape while the prosthetic heart valve is in the radially compressed configuration. This barreled shape, in turn, can apply stress on various components of the prosthetic heart valve frame, such as the frame actuators, and result in plastic deformation and/or buckling of the actuators or other frame components. This challenge is especially problematic in prosthetic heart valve frames having longer actuators with narrower cross sections.

Due to the movement of the frame between the crimped state to the functional state (and vice versa), there is a need for frames for prosthetic heart valves that are flexible to allow for the movement and robust to ensure that the frame functions properly both during and after the implantation procedure.

The prosthetic heart valve frame examples disclosed herein include mechanisms to prevent or mitigate the buckling of the actuation members and other frame components. Various examples disclosed herein can include actuated vertical struts with one or more apertures therein to reduce the rigidity of the actuated vertical struts. Other examples can include heat setting one or more frame elements such as an actuated vertical strut or a commissure window to project either radially inwards or radially outwards from the axis of the prosthetic heart valve frame. Still some examples can include adding additional struts to distribute the elastic forces of compressed frame components more evenly across the entire frame. Any of these examples may be used solely, or in combination with any number of some examples.

In some examples, rod actuators are mounted on a frame of a prosthetic valve. The rod actuators can be rotated in a first direction to radially compress the prosthetic valve to a radially compressed configuration and in a second direction that is opposite to the first direction to radially expand the prosthetic valve to a radially expanded configuration. In some examples, stopper windows are formed in portions of the frame. Stoppers fixedly coupled to the rod actuators are positioned within the stopper windows and can move within the stopper windows as the rod actuators are rotated. In some examples, the stoppers can engage an edge of the stopper windows to facilitate radial compression of the frame. In some examples, apices at an outflow end of the frame can receive head portions of the rod actuators such that the head portions are flush or recessed relative to the outflow end at least when the prosthetic valve is radially expanded to a working diameter.

### The Disclosed Technology and Exemplary Embodiments

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

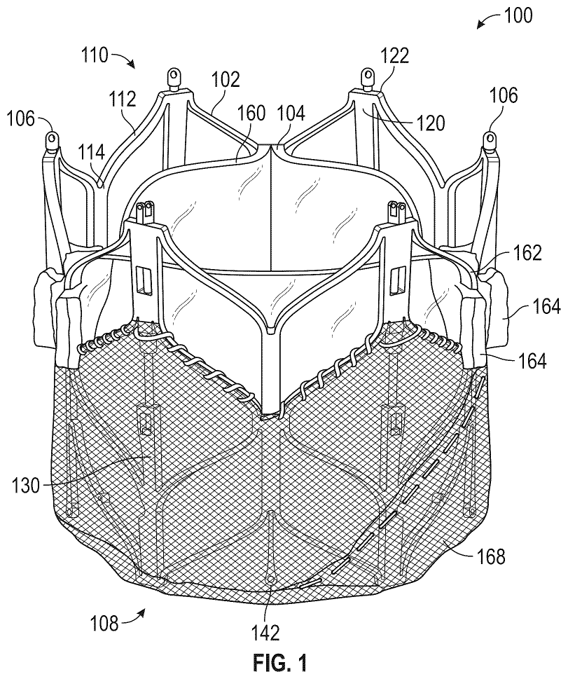

FIG. 1 depicts one example of a prosthetic heart valve which can be radially compressed for delivery through a patient's vasculature and radially expanded to a functional size at a desired implantation location within the patient' body (for example, the native aortic valve). The prosthetic heart valve 100 (also referred to herein as “the prosthetic valve 100”) comprises a frame 102 and a valvular structure 104.

The frame 102 (which can also be referred to as “a stent” or “a support structure”) can be configured to support the valvular structure 104 and for securing the prosthetic heart valve 100 within a native heart valve and/or within another support structure (for example, an anchoring frame (such as a coil) and/or a previously implanted prosthetic heart valve (that is, in a valve-in-valve procedure). The frame 102 can further comprise one or more actuators 106 configured to radially expand or radially compress the frame 102, as described herein.

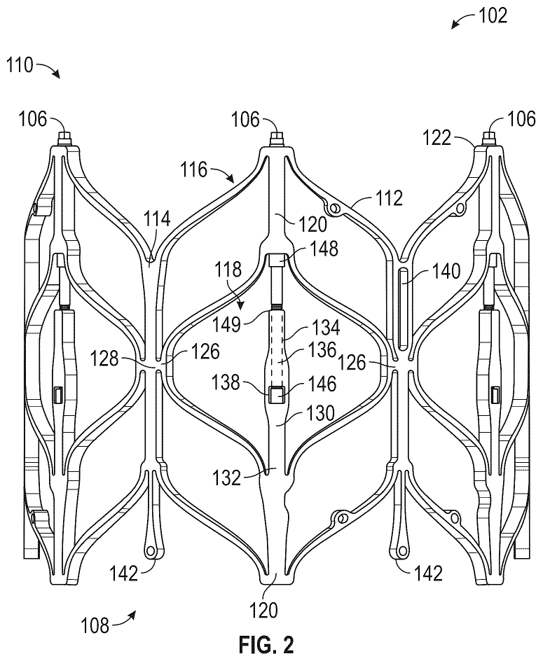

With continued reference to FIGS. 1-2, the frame 102 of the prosthetic heart valve 100 has a first end 108 and a second end 110. In the depicted orientation, the first end 108 of the frame 102 is an inlet end and the second end 110 of the frame 102 is an outlet end. In some examples, the first end 108 of the frame 102 can be the outlet end and the second end 110 of the frame 102 can be the inlet end.

The frame 102 can comprise a plurality of interconnected angled struts 112 and vertical struts 114. In some examples, the angled struts 112 and the vertical struts 114 define a plurality of frame cells. For example, as illustrated in FIG. 2, the angled struts 112 and the vertical struts 114 define a row of six primary cells 116 (which can alternatively be referred to as “outer cells”) and a row of six secondary cells 118 (which can alternatively be referred to as “inner cells) each nested within a respective external cell. The primary cells 116 and the secondary cells 118 can, as illustrated in FIGS. 1 and 2, be connected at their respective axial ends by axial members 120. The primary cells 116 and/or the axial members 120 of the frame 102 can, in some examples, form apices 122 at the first end 108 and the second end 110 of the frame 102.

As illustrated in FIGS. 1 and 2, some of the vertical struts 114 of the frame 102 can be disposed between adjacent pairs of primary cells 116. In some embodiments, the vertical struts 114 can terminate axially inwards from both the first end 108 and the second end 110 of the frame 102. The vertical struts 114 can connect at either axial end to angled struts 112 of the adjacent primary cells 116, such as the two angled struts 112 at each axial end of the vertical struts 114 shown in FIGS. 1 and 2. Accordingly, in some embodiments, the angled struts 112 and the vertical struts 114 can, taken together, define the primary cells 116 of the frame 102, and the primary cells 116 can, as illustrated in FIGS. 1 and 2, have a hexagonal geometry. As shown in FIG. 2, the secondary cells 118 can comprise lateral vertices 126 and can be connected to the vertical struts 114 of the corresponding primary cell 116 by a plurality of lateral members 128, which, in the illustrated embodiments, extend from the lateral vertices 126 of each secondary cell 118 to the nearest corresponding vertical strut 114.

With continued reference to FIG. 2, the frame 102 can also comprise one or more actuated vertical struts 130. The actuated vertical struts 130 can, as shown in FIGS. 1 and 2 be disposed within a secondary cell 118. The actuated vertical struts 130 can be connected at a first end 132 (sometimes called a fixed end 132) to an angled strut 112 or a vertical strut 114 of the secondary cell 118, or to an apex formed by the intersection of two struts 112, 114 of the secondary cell 118. The actuated vertical struts 130 can extend axially from the angled struts 112, the vertical struts 114 or the apex of the secondary cell 118 and can terminate in a second end 134 (called a free end 134 in some examples). The actuated vertical struts 130 can further comprise a bore 136 extending axially from the first end 132 to the second end 134, and configured to receive an actuator, such as actuator 106. In some examples, the actuated vertical strut 130 can further comprise a window 138, configured to receive one or more components of an actuator. Although the actuated vertical struts 130 are described as vertically oriented actuated struts for convenience, it is to be understood that in some examples, the strut may have other nonvertical orientations, such as orientations with a slight angle or curvature relative to an axial axis through the frame.

The frame 102 can further comprise a plurality of leaflet attachment structures. For example, as depicted in FIG. 2, the frame 102 can comprise one or more commissure openings 140 disposed circumferentially between one or more adjacent pairs of the primary cells 116 of the frame 102. The commissure openings 140 can be spaced axially apart from the apices 122 (such as axially inwards) at either the first end 108 or the second end 110 of the frame 102. In the depicted examples, the commissure openings 140 can be bounded on all sides in a “closed” configuration. In some examples, the commissure openings 140 can comprise an open configuration (for example, a U-shaped slot open on one end).

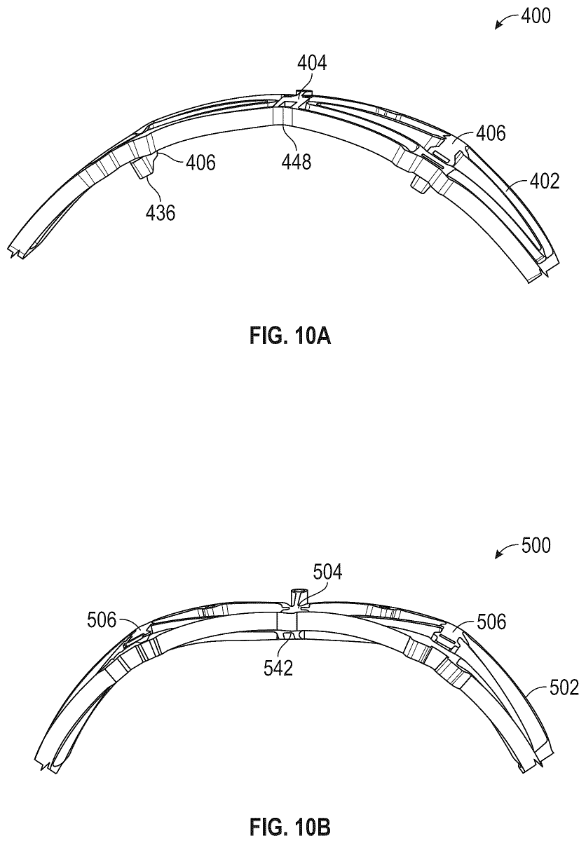

As best illustrated in FIG. 2, the frame 102 can further comprise one or more axially extending suture posts 142. The axially extending suture posts 142 can extend from one or more of the vertical struts 114 as shown in FIGS. 2 and 10. The axially extending suture posts 142 can provide additional locations for affixing the valvular structure 104 or other soft components of the prosthetic heart valve 100.

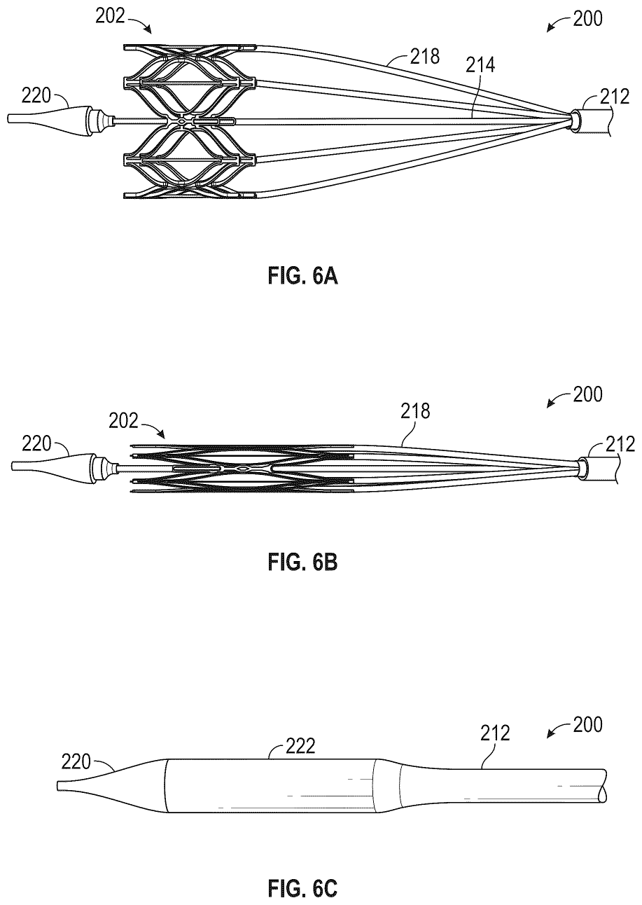

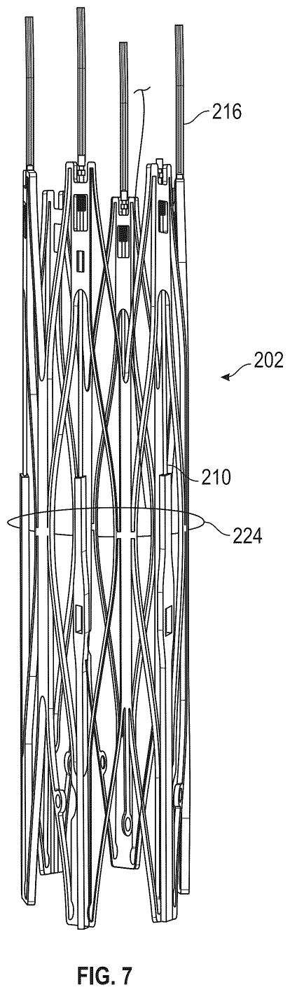

The frame 102 can be configured to move between a plurality of radial configurations, as shown, for example in FIGS. 6A through 7. FIGS. 6B and 7 show a frame 102 in a radially compressed configuration. The depicted configurations are exemplary, and the frame 102 can be expanded or compressed to a lesser or greater extent than depicted. As the frame 102 moves between the various configurations, some of the struts 112, 114 of the frame 102 deflect or pivot relative to each other. For example, the angled struts 112 (which can also be referred to as “diagonal struts”, that is, the non-vertically and non-horizontally oriented struts) deflect relative to the vertically and horizontally oriented struts. In this manner, the frame 102 of the prosthetic heart valve 100 axially elongates when the frame is radially compressed and axially foreshortens when the frame 102 is radially expanded.

While the example prosthetic heart valves described herein include mechanically expandable frames that are expanded by actuators 106, it is to be appreciated that in some examples, different frame expansion mechanisms could be used. For example, self-expanding, partially self-expanding, and balloon expandable frames 102 could be used in place of a mechanically actuated frame as previously described.

Referring again to FIGS. 1-4, the prosthetic heart valve 100 can comprise one or more actuators 106. The actuators 106 are mounted to and spaced circumferentially around the frame 102. In the example illustrated in FIG. 1, the prosthetic heart valve 100 comprises six actuators 106, but it is to be understood that in some examples, fewer actuators (for example, 1-5 actuators) or more actuators (for example, 7-15 actuators) may be used instead. The actuators 106 are configured to, among other things, radially expand and/or radially compress the frame 102.

The actuators 106 can have various forms. For example, in some instances, the actuators 106 can be a rod or shaft. In such instances, the actuators 106 can be formed as separated components from the frame 102, which are then coupled thereto (for example, via welding, adhesive, fasteners, or other means for coupling). Alternatively, the actuators 106 and the frame 102 can be integrally formed as a unitary structure (for example, by forming the frame and actuators from a tube). In some instances, the actuators 106 can be a cable, wire, cord, suture, or other relatively flexible material (that is, compared to a shaft or rod). In such instances, the flexible actuator 106 can be coupled to the frame 102 by tying or looping the actuators 106 around the struts 112, 114 of the frame 102 and/or by coupling the actuator 106 to the frame 102 via a fastener (for example, a grommet), adhesive, and/or other means for coupling.

In some examples, the actuators 106 can be configured for rotational actuation. For example, an actuator 106 may comprise external threads along one or more portions of the actuator 106 (for example, similar to a bolt or screw). As illustrated in FIG. 2, the actuators 106 can comprise a lead screw 149, a nut 146, and a stopper 148. A first end portion of the actuator can be coupled to a first portion (for example, an inlet end portion) of the frame (for example, via the head of the screw) such that the actuator 106 can rotate relative to the first portion of the frame but is axially fixed thereto. In this manner, rotating the actuator 106 in a first direction (for example, clockwise) relative to the frame 102 results in radial expansion of the frame 102 as the first end portion of the frame 102 and the second end portion of the frame move axially toward each other along the threads of the actuator 106. Likewise, rotating the actuator 106 in a second direction (for example, counterclockwise) relative to the frame 102 results in radial compression of the frame 102 as the first end portion of the frame and the second end portion of the frame 102 move axially away from each other along the threads of the actuator 106.

In some examples, the actuators can be configured for linear actuation. In such instances, the actuators 106 comprise fixed end portions fixedly coupled to one portion of the frame (for example, the first end portion) and free end portions movably coupled to another portion of the frame (for example, the second end portion). For example, the fixed end portions of the actuators 106 can be coupled to and/or extend axially from the actuated vertical struts 130 at the inlet end portion of the frame 102, across the primary and second cells and through a lumen traversing the actuated vertical struts 130 at the outlet end portion of the frame 102. The actuator 106 can be used to expand the frame 102 by pulling the actuator 106 toward the outlet end portion of the frame while applying an opposing force on the apices of the outlet end portion of the frame (for example, with a delivery apparatus). These axially-opposing forces together apply a compressive force to the frame and result in radial expansion of the frame. The frame can be radially compressed by reducing tension on the actuators and allowing the elastic properties of the frame to radially compress the frame to its neutral or resting state and/or by an external radially inward force (for example, a crimping device and/or native anatomy within a patient's body).

Each of the actuators can be configured to form a releasable connection with one or more respective actuation shafts of a delivery apparatus. This releasable connection can, for example, include a threaded connection, a plurality of interlocking shafts, and other means of forming a releasable connection. Several examples of releasable connections between the actuators and a delivery apparatus are described herein.

The frame 102 optionally may include a locking mechanism configured to retain the frame 102 in the expanded configuration after the prosthetic heart valve has been radially expanded to the desired diameter. The frame 102 can be configured to freely move between various radially expanded/compressed configurations so long as the locking mechanism is disengaged. When the frame 102 is radially expanded to a desired operational diameter, the locking mechanism can be engaged to prevent further radial expansion and/or contraction of the frame 102.

In some examples, such as examples configured to include rotationally driven actuators, the locking of the prosthetic heart valve 100 can be accomplished by the actuators 106 and the nut 146. In some examples, however, and especially examples using actuators other than rotationally driven actuators, different locking mechanisms, such as locking mechanisms incorporating retention tabs or locking elements may be used instead. Further details regarding prosthetic heart valves, including locking mechanisms and the ways in which locking mechanisms can be incorporated in prosthetic heart valve frames such as frame 102, actuators for radially expanding and compressing prosthetic valves, various frame constructions and methods for assembling prosthetic valves can be found in U.S. Application Nos. 63/085,947, filed Sep. 30, 2020, 63/179,766, filed Apr. 26, 2021, 63/194,285, filed May 28, 2021, and PCT Application No. PCT/US2021/040789, filed Jul. 8, 2021, which are incorporated by reference herein.

The frame 102 can be made of any of various suitable plastically-expandable materials (for example, stainless steel, etc.) or self-expanding materials (for example, Nitinol) as known in the art. When constructed of a plastically-expandable material, the frame 102 (and thus the valve 100) can be crimped to a radially compressed state on a delivery catheter and then expanded inside a patient by an inflatable balloon or equivalent expansion mechanism. When constructed of a self-expandable material, the frame 102 (and thus the valve 100) can be crimped to a radially compressed state and restrained in the compressed state by insertion into a sheath or equivalent mechanism of a delivery catheter. Once inside the body, the valve can be advanced from the delivery sheath, which allows the valve to expand to its functional size.

Suitable plastically-expandable materials that can be used to form the frames disclosed herein (for example, the frame 102) include, metal alloys, polymers, or combinations thereof. Example metal alloys can comprise one or more of the following: nickel, cobalt, chromium, molybdenum, titanium, or other biocompatible metal. In some examples, the frame 102 can comprise stainless steel. In some examples, the frame 102 can comprise cobalt-chromium. In some examples, the frame 102 can comprise nickel-cobalt-chromium. In some examples, the frame 102 comprises a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

When the frame 102 is constructed from a plastically-expandable material, the expansion force required to radially expand the frame is provided by the actuators 106. In some examples, the angled struts 112 and the vertical struts 114 of the frame can be sufficiently rigid to maintain the frame 102 in the radially expanded state against a surrounding native annulus without the use of any locking mechanism.

When the frame 102 is constructed from a shape-memory material (for example, Nitinol), the frame 102 can be configured to self-expand from a radially compressed state to at least a partially radially expanded state. In such cases, the actuators 106 can be used to assist in radially expanding the frame in cooperation with the inherent resiliency of the shape-memory material that urges the frame toward the radially expanded state. For example, the frame 102 can be self-expandable from a radially compressed state to a partially radially expanded state. After the frame reaches the partially radially expanded state, the actuators 106 can be used to further expand the frame 102 from the partially radially expanded state to a fully radially expanded state. After the frame reaches the fully radially expanded state, the actuators 106 can be used to overexpand the frame and dilate the native annulus in which the prosthetic valve is implanted. One or more locking mechanisms, as described herein, can be used to retain the frame in the overexpand state against the forces of the surrounding annulus.

Returning to FIG. 1, the valvular structure 104 of the prosthetic heart valve 100 can be coupled to the frame 102 (for example, directly and/or indirectly via other components such a sealing skirt). The valvular structure 104 is configured to allow blood flow through the prosthetic heart valve 100 from the first end 108 (that is, the inlet end) to the second end 110 (that is, the outlet end) in an antegrade direction and to block blood from flowing through the prosthetic heart valve 100 from the second end 110 to the first end 108 in a retrograde direction. The valvular structure can include various components including a leaflet assembly comprising two or more leaflets 160. For example, the valvular structure 104 in the illustrated example comprises a leaflet assembly having three leaflets 160. It is to be understood, however, that in some examples, the valvular structure 104 could comprise a different number of leaflets.

The leaflets 160 of the prosthetic heart valve 100 can be made of a flexible material. For example, the leaflets 160 can be made from in whole or part, biological material, bio-compatible synthetic materials, or other such materials. Suitable biological material can include, for example, bovine pericardium, equine pericardium, porcine pericardium, and/or pericardium from other sources.

The leaflets 160 can be arranged to form commissures 162. The commissures 162 can, for example, be mounted to the frame at the commissure windows 140, as illustrated in FIG. 1. For example, each leaflet 160 can have two commissure tabs 164 on opposite sides of the leaflet 160. Each commissure tab 164 can be paired with an adjacent commissure tab 164 of an adjacent leaflet to form a respective commissure 162. Each pair of commissure tabs 164 can be coupled to a corresponding vertical strut 114 at a commissure window 140, such as by sutures or other fastening means. Each commissure 162 can include one or more reinforcing members, such as fabric reinforcing members, that are sutured to the commissure tabs 164 and/or the vertical struts 114 to reinforce the connection between the commissure tabs 164 and the vertical struts 114.

The inlet or cusp edge portions of the leaflets 160 can be coupled to the frame 102 via various techniques and/or mechanisms. For example, the cusp edge portions of the leaflets 160 can be sutured directly to selected angled struts 112 or vertical struts 114 of primary cells 116 located at the first end 108 (that is, inlet end) of the prosthetic heart valve. Alternatively, the cusp edge portions of the leaflets 160 can be sutured to an inner skirt (for example, a fabric skirt, not shown), which in turn can be sutured to selected angled struts 112 or vertical struts 114 of primary cells 116 located at the first end 108 (that is, the inlet end) of the prosthetic heart valve. The inlet portions of the leaflets 160 can also, in some examples, be coupled to the one or more axially extending suture posts 142 extending from selected vertical struts 114.

With continued reference to FIG. 1, the valvular structure 104 can further include an outer skirt or sealing member 168 disposed around the exterior of the frame 102. The outer skirt 168 can be made of any suitable biocompatible and flexible material, including materials suitable for leaflets 160, or synthetic material, such as any suitable biocompatible fabric (for example, polyethylene terephthalate (PET) fabric). The outer skirt 168 can be attached to the frame 102 by means of sutures, fabric, adhesive and/or other means for mounting, and in certain examples can be attached to the angled struts 112 and/or vertical struts 114 of primary cells 116 located at the first end 108 (that is, the inlet end) of the prosthetic heart valve. The outer skirt 168 can be configured to improve the seal between the prosthetic heart valve 100 and the native heart valve in which the prosthetic heart valve has been implanted.

The skirt can be wholly or partly formed of any suitable biological material, synthetic material (for example, any of various polymers), or combinations thereof. In some examples, the skirt can comprise a fabric having interlaced yarns or fibers, such as in the form of a woven, braided, or knitted fabric. In some examples, the fabric can have a plush nap or pile, Exemplary fabrics having a plus nap or pile include velour, velvet, velveteen, corduroy, terrycloth, fleece, etc. In some examples, the skirt can comprise a fabric without interlaced yarns or fibers, such as felt or an electrospun fabric. Exemplary materials that can be used for forming such fabrics (with or without interlaced yarns or fibers) include, without limitation, polyethylene (PET), ultra-high molecular weight polyethylene (UHMWPE), polytetrafluoroethylene (PTFE), expanded polytetrafluoroethylene (ePTFE), polyamide etc. In some examples, the skirt can comprise a non-textile or non-fabric material, such as a film made from any of a variety of polymeric materials, such as PTFE, PET, polypropylene, polyamide, polyetheretherketone (PEEK), polyurethane (such as thermoplastic polyurethane (TPU)), etc. In some examples, the skirt can comprise a sponge material or foam, such as polyurethane foam. In some examples, the skirt can comprise natural tissue, such as pericardium (for example, bovine pericardium, porcine pericardium, equine pericardium, or pericardium from other sources).

Further details regarding prosthetic heart valves, including the valvular structure 104 and manner in which the valvular structure 104 can be coupled to the frame 102 of the prosthetic heart valve 100, can be found in U.S. Pat. Nos. 6,730,118, 7,393,360, 7,510,575, 7,993,394, 8,652,202, and 9,393,110, U.S. Publication No. 2018/0325665, and U.S. Application No. 63/138,890, filed Jan. 19, 2021, which are incorporated by reference herein.

The examples of prosthetic heart valves described herein may be configured to be implanted in the vasculature of a patient by a delivery apparatus. A suitable delivery apparatus may comprise an elongated shaft configured to pass through the vasculature of a patient, one or more delivery actuators to manipulate a prosthetic heart valve within the patient's body, and a control mechanism by which a physician may control the actuators. Some examples of a delivery apparatus may further include a restraining mechanism configured to retain the prosthetic heart valve in a compressed configuration.

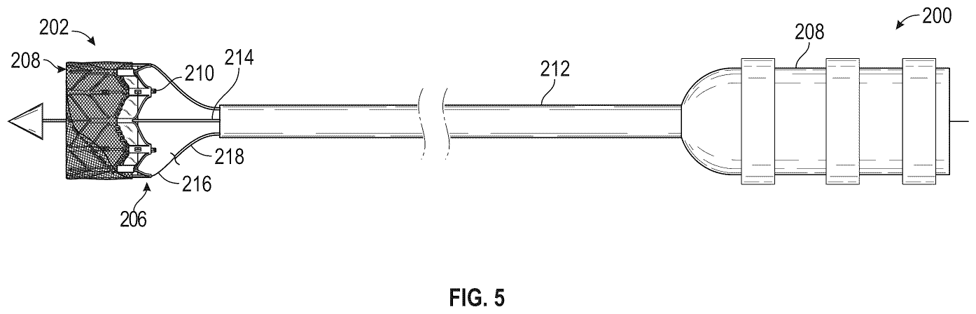

FIG. 5 illustrates a delivery apparatus 200, according to one example, designed to advance a prosthetic heart valve 202 through a patient's vasculature and/or to deliver the prosthetic heart valve 202 to an implantation site (for example, native heart valve) within a patient's body. The prosthetic heart valve 202 can be mounted on, retained within, and/or releasably coupled to a distal end portion of the delivery apparatus 200. The prosthetic valve 202 can represent the prosthetic heart valve 100 previously described herein and illustrated in FIG. 1.

The prosthetic heart valve 202 can include a distal end 204 (which can be the inlet end of the prosthetic heart valve 202, such as when the prosthetic heart valve 202 is configured to replace a defective aortic valve when delivered transfemorally) and a proximal end 206 (which can be the outlet end of the prosthetic heart valve 202, such as when the prosthetic heart valve 202 is configured to replace a defective aortic valve when delivered transfemorally), wherein the proximal end 206 is positioned closer to a handle 208 of the delivery apparatus 200 than the distal end 204, and wherein the distal end 204 is positioned farther from the handle 208 than the proximal end 206. It is to be understood that in some examples, such as when the prosthetic heart valve 202 is implanted in a different location in the vasculature of the patient, the proximal end 206 can alternatively be an inlet end of the prosthetic heart valve 202 and the distal end 204 can be an outlet end of the prosthetic heart valve 202. The prosthetic heart valve 202 can also include one or more actuators 210, extending from the distal end 204 to the proximal end 206, as has been discussed herein.

The delivery apparatus 200 in the illustrated example generally includes the handle 208, a first shaft 212 (an outer shaft in the illustrated example) extending distally from the handle 208, a second shaft 214 (an inner shaft in the illustrated example) extending distally from the handle 208 through the first shaft 212, one or more delivery system actuators 216 extending distally through the outer shaft 212, and one or more support tubes (sometimes called support members) 218 that can extend distally through the outer shaft 212 and can abut the proximal end 206 of the prosthetic heart valve 202. The delivery apparatus 200 can further include a nose cone 220 connected to the distal end portion of the second shaft 214.

Each delivery system actuator 216 can have a distal end connected to an actuator 210 of the prosthetic heart valve 202. Each of the delivery system actuators 216 can extend through a respective support tube 218 and together can define a respective actuator assembly that can extend through the outer shaft 212 to the handle 208. In alternative examples, the delivery system actuators 216 and the support tubes 218 need not be co-axial with respect to each and instead can extend side-by-side through the shaft.

When the prosthetic heart valve includes linear actuators 210, the delivery system actuators 216 and/or the support tubes 218 can be configured to radially expand the prosthetic heart valve 202 by bringing the ends 204, 206 of the prosthetic heart valve 202 closer together (that is, squeezing the prosthetic heart valve 202 axially) thereby axially foreshortening and radially expanding the prosthetic heart valve 202. As one example, the delivery system actuators 216 can be configured to be actuated to provide a proximally directed (for example, pulling) force to the actuators 210 of the prosthetic heart valve 202 while the one or more support tubes 218 can be configured to provide a countervailing distally directed (for example, pushing) force to the proximal end 206 of the prosthetic heart valve 202. The actuators 210, in turn, may transmit the force to the distal end 204 of the prosthetic heart valve 202. In one such example, a physician can pull the delivery system actuators 216 to provide the proximally directed force to the distal end 204 of the prosthetic heart valve 202, while simultaneously gripping, holding, and/or pushing the handle 208 to provide the countervailing distally directed force to the proximal end 206 of the prosthetic heart valve 202.

When the prosthetic heart valve includes rotationally-driven actuators 210, the delivery system actuators 216 can be configured to apply a rotational force to the actuators 210. In such examples, the actuators 210 may have a first threaded end configured to connect with a corresponding threaded end of a delivery system actuator 216. When the delivery system actuator 216 is rotated in a first rotational direction, the actuator 210 can exert an axial force in the proximal direction on the distal end 204 of the prosthetic heart valve 202, thereby axially foreshortening and radially expanding the prosthetic heart valve 202. When the delivery system actuator 216 is rotated in a second rotational direction opposite to the first rotational direction, the actuator 210 can exert an axial force in the distal direction on the distal end 204 of the prosthetic heart valve 202, thereby axially extending and radially contracting the prosthetic heart valve 202. In such an example, a physician can rotationally manipulate the actuators 210 of the prosthetic heart valve 202 to radially expand or contract the prosthetic heart valve 202 to a desired diameter.

As described herein, the delivery system actuators 216 can cooperate with a locking element on the prosthetic heart valve 202 to retain the prosthetic heart valve in a radially expanded state.

Although two pairs of delivery system actuators 216 and support tubes 218 are shown in FIG. 5, it should be understood that the delivery apparatus 200 can include more or less than three delivery system actuators 216 and/or three support tubes 218, in some examples. As just one example, the delivery apparatus 200 can include six delivery system actuators 216 and/or six support tubes 218. In some examples, a greater or fewer number of delivery system actuators 216 and/or support tubes 218 can be present, such as four, five, seven, and/or eight delivery system actuators 216 and/or four, five, seven, and/or eight support tubes 218. In some examples, the delivery apparatus 200 can include equal numbers of delivery system actuators 216 and support tubes 218. However, in some examples, the delivery apparatus 200 can include a different number of delivery system actuators 216 and support tubes 218.

Examples of the delivery apparatus disclosed herein may further include a restraining mechanism configured to retain the prosthetic heart valve in a compressed state. The restraining mechanism may be releasably attached to the prosthetic heart valve while the prosthetic heart valve is being advanced through the vasculature of the patient and/or being positioned at the desired implantation site and may be detached once the prosthetic heart valve has been positioned in the desired location.

In some examples, illustrated in FIG. 6C, the restraining mechanism is a delivery capsule 222 (which may also be referred to as a “sheath”) configured to surround and restrain the prosthetic heart valve in a radially compressed state. The delivery capsule 222 can extend from the distal end of the outer shaft 212 of the delivery apparatus 200, or it can be an integral component of the outer shaft 212. When delivery apparatus 200 advances the prosthetic heart valve 202 to the implantation site, the delivery capsule 222 can be retracted in the proximal direction (that is, towards the handle of the delivery apparatus) to deploy the prosthetic heart valve 202. When the prosthetic heart valve 202 is deployed from the delivery capsule, as shown in FIGS. 6A and 6B, the prosthetic heart valve may be expanded to the partially radially expanded state (FIG. 6B) or to the fully radially expanded state (FIG. 6A).

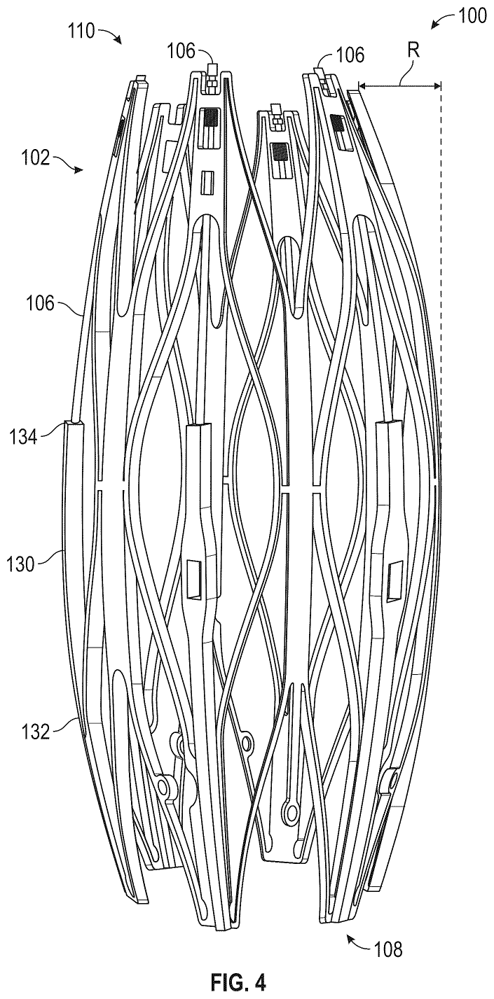

In lieu of or in addition to a delivery capsule, as illustrated in FIG. 7, the restraining mechanism can include an adjustable loop or lasso 224 circumferentially disposed around the exterior of the prosthetic heart valve 202. The adjustable loop 224 is configured to allow the prosthetic heart valve 202 to expand to the partially radially expanded state (FIG. 4B) or to the fully radially expanded state (FIG. 4A) by introducing slack in the loop 224, allowing the loop 224 to increase in diameter.

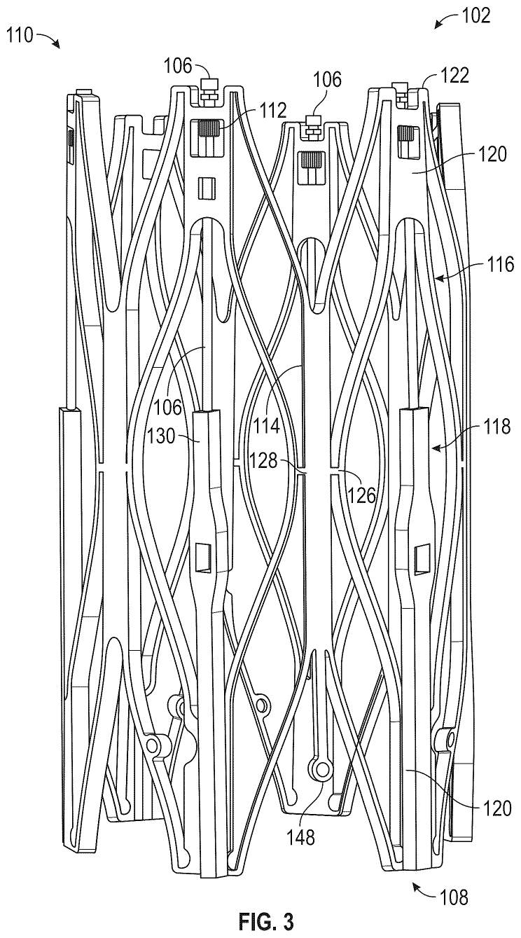

Also disclosed herein are various examples of prosthetic heart valves having frame elements configured to reduce the bending or buckling of the actuators, such as actuators 106. When the prosthetic heart valve 100 is in the radially compressed configuration, the frame 102 can tend to compress more at the first end (sometimes called the distal end) 108 and the second end (sometimes called the proximal end) 110 than at the axial midpoint of the frame, in a phenomenon sometimes known as “barreling”, shown in FIG. 4. Without being limited to any particular theory, it is currently believed that this difference in contraction is due to the higher radial strength of the frame 102 closer to the axial midpoint. The actuated vertical struts 130, in some examples, can be attached to the frame at only the first end 132 (that is, at the fixed end 132), as shown in FIGS. 3 and 4. Thus, the frame 102 may exert a compressive force on the fixed end 132 of the actuated vertical strut 130 as the prosthetic heart valve 100 is radially compressed, while exerting reduced compressive force on the second end 134 (that is, the free end) of the actuated vertical strut 130. This may cause the actuated vertical strut to cantilever out from the frame 102 as the prosthetic heart valve 100 is radially compressed with a radial distance, R, between the second end 134 of the actuated vertical strut 130 and the ends 108, 110 of the frame 102.

As shown in FIG. 4, the actuators 106 can be in contact with the frame 102 at or near the second end (sometimes called the proximal end) 110, and at the second end 134 of the actuated vertical strut 130. Because the second end 134 of the actuated vertical strut 130 may be radially compressed to a lesser degree than either the first end (sometimes called the distal end) 108 or the second end (sometimes called the proximal end) 110 of the frame 102, or the actuated vertical strut 130, the frame 102 can impart a bending moment on the actuators 106, causing the actuators 106 to bow radially outwards relative to the frame 102, as illustrated in FIG. 4. In some cases, this outwards radial bowing of the actuators 106 can cause the actuators 106 to buckle (that is, to plastically deform from a linear configuration). Bowed actuators may subsequently pose several problems during the implantation procedure, particularly during any steps requiring the expansion of the prosthetic heart valve from the radially compressed state to a partially radially expanded state or a fully radially expanded state.

The buckling of actuators can be addressed in several ways in the various examples disclosed herein. In some examples, the frame of the prosthetic heart valve can include actuated vertical struts having one or more apertures. In some examples, the frame of the prosthetic heart valve can include actuated vertical struts that are heat set radially away from the outer circumference of the frame. In yet some examples, the frame of the prosthetic heart valve can include commissure openings that are heat set away from the outer circumference of the frame. In still some examples, the frame can include one or more lateral struts that connect the actuated vertical struts to the cells of the frame. It is to be appreciated that any of these frame elements may be used alone, or in conjunction with any or all of the other frame elements disclosed herein. The various example prosthetic heart valves disclosed herein can, therefore, reduce or eliminate the problems associated with the buckling of the actuators, as will be discussed herein.

In some examples, the actuated vertical strut (such as actuated vertical strut 130 previously described) of a prosthetic heart valve frame (such as frame 102 previously described) can include one or more apertures set in the length of the actuated vertical strut. These apertures can serve to reduce the structural rigidity of the actuated vertical strut (that is, the inclusion of the apertures can reduce the flexural modulus of the actuated vertical strut, increasing its tendency to flex under bending forces). When prosthetic heart valves according to the present disclosure are held in the radially compressed configuration, the barreling of the frame illustrated in FIG. 4 will cause the actuator to impart a radially inward bending force on the free end of the actuated vertical strut. In example prosthetic heart valves having an actuated vertical strut with reduced rigidity (that is, increased flexibility), the bending moment applied to the free end of the actuated vertical strut by the contact between the actuated vertical strut and the actuator may result in a greater radially inwards deflection of the actuated vertical strut. As a result, the radial distance between the free end of the actuated vertical strut and the ends of the frame may be smaller in such examples, and the corresponding bowing of the actuator can be reduced.

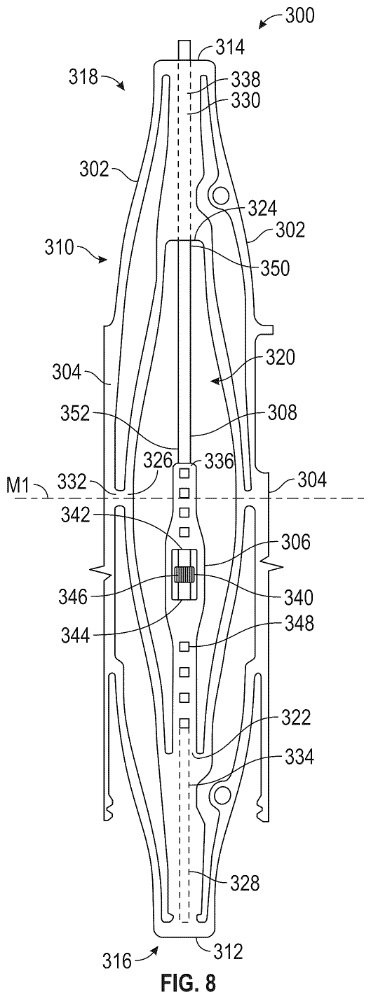

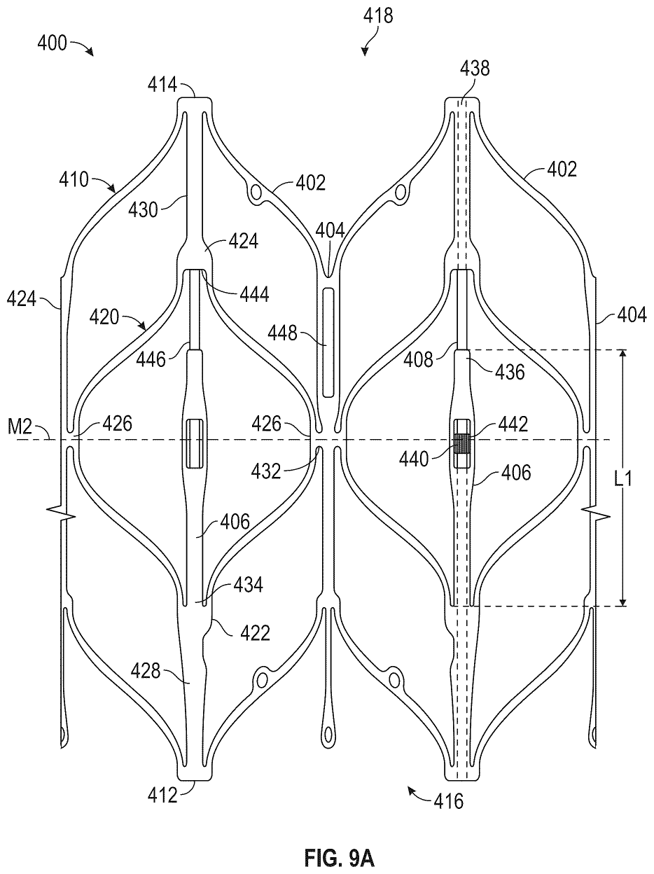

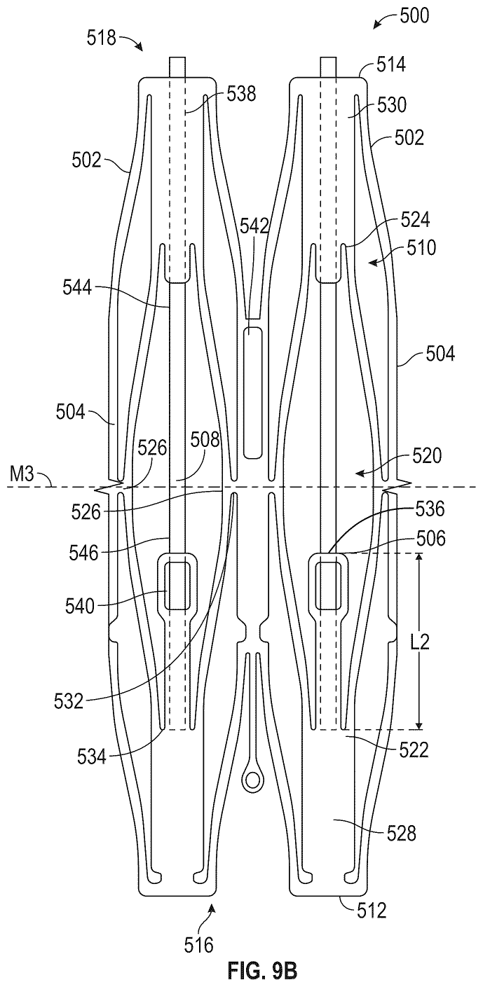

FIG. 8 illustrates a portion of a frame 300 which can include an actuated vertical strut having a plurality of apertures for increased flexibility. As shown in FIG. 8, the frame 300 comprises a plurality of angled struts 302, a plurality of vertical struts 304, and one or more actuated vertical struts 306, and can be configured to receive one or more actuators, such as actuators 308. The frame 300 can comprise a plurality of portions similar or substantially identical to the one shown in FIGS. 9B and 10B arranged adjacent to each other to form an annular frame for a prosthetic heart valve. In some examples, the frame 300 can comprise six outer cells, but in some examples, a greater or lesser number of outer cells such as 4, 5, 7, 8, 9, 10, 11, or 12 outer cells can make up frame 400.

As shown in FIG. 8, the frame 300 can comprise a plurality of interconnected angled struts 302 and vertical struts 304 that form a plurality of outer cells 310 (sometimes called primary cells 310). Each outer cell 310 has an outer distal apex 312 and an outer proximal apex 314. In some examples, such as the one illustrated in FIG. 8, one or more outer distal apices 312 may define an inlet end 316 of the frame 300 and one or more outer proximal apices may define an outlet end 318 of the frame 300. It is to be understood that in some examples, however, the outer distal apices 312 may define the outlet end 318 of the frame 300 and the outer proximal apices 314 may define the inlet end 316 of the frame. The outer cells 310 can also comprise two vertical struts 304, and each outer cell 310 can be connected to two adjacent outer cells 310 along shared vertical struts 304 to form the frame 300.

With continued reference to FIG. 8, the plurality of angled struts 302 and vertical struts 304 can also form an inner cell 320 (sometimes called a secondary cell 320). The inner cell 320 can have an inner distal apex 322 and an inner proximal apex 324, as well as two medial vertices 326. In some examples, such as that shown in FIG. 8, a first axial member 328 can extend from the inner distal apex 322 of the inner cell 320 to the outer distal apex 312 of the corresponding outer cell 310, and a second axial member 330 can extend from the inner proximal apex 324 of the inner cell 320 to the outer proximal apex 314 of the corresponding outer cell 310 to connect the inner cells 320 to the corresponding outer cells 310. In some examples, lateral members 332 can extend from the medial vertices 326 of the inner cells to the vertical struts 304 of the corresponding outer cells. While FIG. 8 shows an inner cell connected to a corresponding outer cell by two axial members and two lateral members, it is to be understood that one or more of these connecting members may, in alternative examples, be absent from the frame 300.

As shown in FIG. 8, the frame 300 can also include an actuated vertical strut 306. The actuated vertical strut 306 can have a fixed end 334 and a free end 336. The actuated vertical strut 306 can attach at the fixed end 334 to the inner distal apex 322 of a secondary cell 320 and can extend axially from the inner distal apex 322 towards the inner proximal apex 324 of the secondary cell 320 while leaving the free end 336 unattached to any other component of the frame. For example, the actuated vertical strut can be cantilevered. In some examples, such as that shown in FIG. 8, the free end 336 of the actuated vertical strut 306 can extend past an axial midpoint M1 of the secondary cell 320 when the frame 300 is in the radially compressed or partially radially expanded configuration. It is to be appreciated, however, that in alternative examples, the actuated vertical strut 306 can be shorter than shown in FIG. 8, such that the free end 336 of the actuated vertical strut 306 is axially aligned with the axial midpoint M1 of the inner cell 320, or stops axially short of the axial midpoint M1 of the inner cell 320. It is also to be appreciated that in some examples, the actuated vertical strut 306 can be longer than shown in FIG. 8. Although the actuated struts 306 are described as vertically oriented actuated struts for convenience, it is to be understood that in some examples, the strut may have other nonvertical orientations, such as orientations with a slight angle or curvature relative to an axial axis through the frame.

In some examples, as shown in FIG. 8, a channel 338 can extend through the first axial member 328, the second axial member 330 and the actuated vertical strut 306. The channel 338 can be configured to admit the actuator 308, which can extend from the inlet end 316 of the frame 300 towards the outlet end 318 of the frame 300. The actuator 308 can be configured, as previously discussed, to draw the inlet end 316 and the outlet end 318 of the frame 300 closer together, thereby axially foreshortening and radially expanding (that is, from a radially compressed configuration to a partially radially expanded configuration or from a partially radially expanded configuration to a fully radially expanded configuration) the frame 300. Likewise, the actuator 308 can be configured to push the inlet end 316 and the outlet end 318 of the frame 300 further apart, thereby axially extending and radially contracting (that is, from a fully radially expanded configuration to a partially radially expanded configuration or from a partially radially expanded configuration to a radially compressed configuration) the frame 300. In some examples, the channel 338 can also be configured to admit a delivery system actuator, such as delivery system actuator 216.

In examples in which the prosthetic heart valve includes actuators 308 configured for rotatable actuation, the actuated vertical strut 306 can further comprise a window 340. The window 340 can have a proximal end 342 and a distal end 344, and can be configured to accommodate a component of the actuator 308, such as an actuation nut 346, which rests within the window 340 and is threadably attached to a portion of the actuator 308. In such examples, because the actuation nut 346 cannot move in the proximal direction (that is, towards the outlet end 318 of the frame 300) past the proximal end 342 or in the distal direction (that is, towards the inlet end 316 of the frame 300) past the distal end 344 of the window 340, and because the nut is threadably attached to the actuator 308, the nut may limit the axial range of motion of the actuator 308.

With continued reference to FIG. 8, the actuated vertical strut 306 can also include one or more apertures 348 disposed between the fixed end 334 and the free end 336. The apertures 348 can reduce the flexural rigidity of the actuated vertical strut 306, causing the axially extending vertical strut to more easily flex radially inwards or outwards from a neutral position. Because the actuated vertical strut 306 having one or more apertures 348 is less rigid than an actuated vertical strut omitting the apertures, but can be otherwise identical, the actuated vertical strut 306 can deflect radially inwards to a greater degree when the frame 300 is in the radially compressed configuration. As a result, in such examples, the radial displacement between the free end 336 of the actuated vertical strut 306 and the ends 316, 318 of the frame 300 can be less than such a radial displacement in a frame having an actuated vertical strut omitting such apertures (that is, an actuated vertical strut with greater stiffness). In turn, this can reduce the radial displacement (shown in FIG. 4 as R) between an end portion 350 and a central portion 352 of the actuator 308 when the frame 300 is in a radially compressed configuration. This can reduce the degree of bending of the actuator 308 when the frame 300 is in a radially compressed configuration, and can reduce the likelihood of plastic deformation and/or buckling of the actuator 308.

In some examples, elements of a frame (such as frame 102) can be plastically deformed or heat set in a deformed configuration suitable for protecting any actuators connected to the frame from bending and/or buckling when the frame is in the compressed configuration. In some examples, the actuated vertical struts can be plastically deformed or heat set to extend radially inwards from the other struts of the frame when the frame is in the radially expanded configuration, resulting in a smaller radial displacement between the actuated vertical struts and the ends of the frame when the frame is in the radially compressed configuration. In some examples, frame struts having commissure openings can be plastically deformed or heat set to extend radially inwards from adjacent struts of the frame when the frame is in the radially expanded configuration, tending to exert force on adjacent actuated struts and reducing the radial displacement between the actuated vertical struts and the ends of the frame when the frame is in the radially compressed configuration.

FIGS. 9A and 10A illustrate sections of one example of a frame 400 having heat set or plastically deformed components, in a radially compressed or partially radially expanded configuration. As shown in FIG. 9A, the frame 400 comprises a plurality of angled struts 402, a plurality of vertical struts 404, and one or more actuated vertical struts 406, and can be configured to receive one or more actuators, such as actuators 408. The frame 400 can comprise a plurality of portions similar or substantially identical to the one shown in FIGS. 9A and 10A arranged adjacent to each other to form an annular frame for a prosthetic heart valve.

As shown in FIG. 9A, the plurality of angled struts 402 and the plurality of vertical struts 404 can form a plurality of outer cells 410 (sometimes called primary cells 410). The outer cells 410 can each have an outer distal apex 412 and an outer proximal apex 414. In some examples, the outer distal apices 412 of the plurality of outer cells 410 can define an inlet end 416 of the frame 400 and the outer proximal apices 414 of the plurality of outer cells 410 can define an outlet end 418 of the frame 400. It is to be understood, however, that in some examples, the outer distal apices 412 may define the outlet end 418 of the frame 400 and the outer proximal apices 414 can define an inlet end 416 of the frame 400. As shown in FIG. 9, each outer cell 410 can be connected to an adjacent outer cell 410 along a vertical strut 404. In some examples, each outer cell 410 can be connected in this way to two adjacent outer cells and arranged in a circular formation to form an annular frame 400. In some examples, the frame 400 can comprise six outer cells, but in some examples, a greater or lesser number of outer cells such as 4, 5, 7, 8, 9, 10, 11, or 12 outer cells can make up frame 400.

With continued reference to FIG. 9A, the angular struts 402 can also form a plurality of inner cells 420 (sometimes called secondary cells 420). The inner cells 420 can each have an inner distal apex 422 and an inner proximal apex 424, as well as two medial vertices 426. Each inner cell 420 can be disposed within a corresponding outer cell 410, as illustrated in FIG. 9A. In some examples, such as that shown in FIG. 9A, a first axial member 428 can extend from the inner distal apex 422 of the inner cell 420 to the outer distal apex 412 of the corresponding outer cell 410, and a second axial member 430 can extend from the inner proximal apex 424 of the inner cell 420 to the outer proximal apex 414 of the corresponding outer cell 410 to connect the inner cells 420 to the corresponding outer cells 410. In some examples, lateral members 432 can extend from the medial vertices 426 of the inner cells to the vertical struts 404 of the corresponding outer cells. While FIG. 9A shows an inner cell connected to a corresponding outer cell by two axial members and two lateral members, it is to be understood that one or more of these connecting members may, in alternative examples, be absent from the frame 400.

As shown in FIG. 9A, the actuated vertical strut 406 can have a first end (sometimes called a fixed end) 434 and a second end (sometimes called a free end) 436. The actuated vertical strut 406 can attach at the fixed end 436 to the inner distal apex 422 of an inner cell 420, and can extend axially from the inner distal apex 422 towards the inner proximal apex 424 of the inner cell 420. For example, the actuated vertical strut can be cantilevered. In some examples, such as that shown in FIG. 9A, the length L1 of the actuated vertical strut 406 is such that the free end 436 can extend past an axial midpoint M2 of the inner cell 420 when the frame is in the radially compressed or partially radially expanded configuration. It is to be appreciated, however, that in alternative examples, the length L1 of the actuated vertical strut 406 can be shorter than that shown in FIG. 9A, such that the free end 436 terminates at the axial midpoint M2 of the inner cell 420 or between the axial midpoint M2 and the inner distal apex 422 when the frame 400 is in the radially compressed or partially radially expanded configuration. It is also to be appreciated that in some examples, the length L1 of the actuated vertical strut 406 can be longer than that shown in FIG. 9A. Although the actuated struts 406 are described as vertically oriented actuated struts for convenience, it is to be understood that in some examples, the strut may have other nonvertical orientations, such as orientations with a slight angle or curvature relative to an axial axis through the frame.

With continued reference to FIG. 9A, a channel 438 can extend through the second axial member 430 and the actuated vertical strut 406. In some examples, the channel 438 can also extend through the first axial member 428. The channel 438 can be configured to admit at least a portion of the actuator 408, which can extend from the outlet end 418 towards the inlet end 416 of the frame 400. The channel 438 can also be configured to admit at least a portion of a delivery system actuator, such as delivery system actuator 216 shown in FIGS. 5 and 7. In some examples, such as that illustrated in FIG. 9A, the actuated vertical strut 406 can also include a window 440. The window 440 can be configured to accommodate various components of the actuator 408. In some examples, such as those having a rotatably-driven actuator, the window 440 can contain an actuator nut 442 configured to limit the axial range of motion of the actuator 408 as previously discussed.

The frame 400 can also include one or more commissure openings 448, as shown in FIGS. 9A and 10A. The one or more commissure openings can be formed in one or more non-actuated struts, such as vertical struts 404, and can be configured to receive one or more leaflets of a valvular structure. In some examples, such as that illustrated in FIG. 9A, the commissure opening 448 can be closed. Closed commissure openings 448 may advantageously permit a more secure attachment of the leaflets of the valvular structure to the frame 400. However, it is also to be appreciated that the commissure opening 448 may be open, for example at the end of the commissure opening closest to either the inlet end or the outlet end of the frame 400. A commissure opening 448 with an open configuration may, for example, allow an easier attachment of the valvular structure to the frame 400. While FIG. 9A shows the commissure opening 448 formed in a portion of the vertical strut 404 disposed towards the outlet end of the frame 400, it is to be appreciated that in some examples, the commissure opening 448 can be formed a portion of a vertical strut 404 disposed towards the inlet end of the frame 500.

Referring now to FIG. 10A, the actuated vertical strut 406 can extend radially inwards from the other components of the frame 400. As illustrated in FIG. 10A, the free end 436 of the actuated vertical strut 406 can, in this way, be disposed radially inwards of the body of frame 400. In some examples, the radially inwards extension of the actuated vertical strut 406 can be accomplished by plastically deforming the actuated vertical strut 406 radially inwards relative to the frame 400, and then applying a shape setting heat treatment to shape set (sometimes called heat set) the actuated vertical strut in this radially deflected configuration. It is to be appreciated, however, that in some examples, the shape setting heat treatment may be omitted, and the plastic deformation of the actuated vertical strut 406 can be sufficient to retain the actuated vertical strut in a configuration in which it extends radially inwards from the rest of the frame.

In examples of frames having an actuated vertical strut that is heat set inwards relative to the body of the frame, the radially inwards disposition of the free end of the actuated vertical strut while the frame is in the radially expanded configuration can prevent or minimize the bending any actuator connected with the frame when the frame is in the radially compressed configuration. For example, the free end 436 of the actuated vertical strut 406 can have a reduced radial displacement relative to the inlet end 416 and the outlet end 418 of the frame 400 when the frame 400 is in the compressed configuration. In turn, this can reduce the radial displacement between an end portion 444 and a center portion 446 of the actuator 408, illustrated in FIG. 9A, when the frame is in the radially compressed configuration. This reduced radial displacement can reduce the degree of bending of the actuator 408 when frame 400 is in a radially compressed configuration, and can tend to reduce the likelihood of plastic deformation or buckling of the actuator 408.

FIGS. 9B and 10B illustrate a section of some examples frame 500 having heat set or plastically deformed components, in a radially compressed or partially radially expanded configuration. As shown in FIG. 9B, the frame 500 comprises a plurality of angled struts 502, a plurality of vertical struts 504, and one or more actuated vertical struts 506, and can be configured to receive one or more actuators, such as actuators 508. The frame 500 can comprise a plurality of portions similar or substantially identical to the one shown in FIGS. 9B and 10B arranged adjacent to each other to form an annular frame for a prosthetic heart valve.

As shown in FIG. 9B, the plurality of vertical angled struts 502 and the plurality of vertical struts 504 can form a plurality of outer cells 510 (sometimes called primary cells 510). The outer cells 510 can each have an outer distal apex 512 and an outer proximal apex 514. In some examples, the outer distal apices 512 of the plurality of outer cells 510 can define an inlet end 516 of the frame 500 and the outer proximal apices 514 of the plurality of outer cells 510 can define an outlet end 518 of the frame 500. It is to be understood, however, that in some examples, the outer distal apices 512 may define the outlet end 518 of the frame 500 and the outer proximal apices 514 can define an inlet end 516 of the frame 500. As shown in FIG. 9B, each outer cell 510 can be connected to an adjacent outer cell 510 along a vertical strut 504. In some examples, each outer cell 510 can be connected in this way to two adjacent outer cells 510 and arranged in a circular formation to form an annular frame 500. In some examples, the frame 500 can comprise six outer cells, but in some examples, a greater or lesser number of outer cells such as 4, 5, 7, 8, 9, 10, 11, or 12 outer cells can make up frame 400.

With continued reference to FIG. 9B, the angular struts 502 also can form a plurality of inner cells 520 (sometimes called secondary cells 520). The inner cells 520 can each include an inner distal apex 522 and an inner proximal apex 524, as well as two medial vertices 526. Each inner cell 520 can be disposed within a corresponding outer cell 510, as illustrated in FIG. 9B. In some examples, such as the one illustrated in FIG. 9B, a first axial member 528 can extend from the inner distal apex of the inner cell 520 to the outer distal apex 512 of the corresponding outer cell 510, and a second axial member 530 can extend from the inner proximal apex 524 of the inner cell 520 to the outer proximal apex 514 of the corresponding outer cell 510 to connect the inner cells 520 to the corresponding outer cells 510. In some examples, lateral members 532 can extend from the medial vertices 526 of the inner cells to the vertical struts 504 of the corresponding outer cells. While FIG. 9A shows an inner cell connected to a corresponding outer cell by two axial members and two lateral members, it is to be understood that one or more of these connecting members may, in alternative examples, be absent from the frame 500.

As shown in FIG. 9B, the actuated vertical strut 506 can have a first end (sometimes called a fixed end) 534 and a second end (sometimes called a free end) 536. The actuated vertical strut 506 can attach at the fixed end 534 to the inner distal apex 522 of an inner cell 520, and can extend axially from the inner distal apex 522 towards the inner proximal apex 524 of the inner cell 520. For example, the actuated vertical strut can be cantilevered. In some examples, such as that shown in FIG. 9B, the length L2 of the actuated vertical strut 506 is such that the free end 536 can terminate short of the axial midpoint M3 of the inner cell 520 when the frame is in the radially compressed or partially radially expanded configuration. It is to be appreciated, however, that in alternative examples, the length L2 of the actuated vertical strut 506 can be longer than that shown in FIG. 9B, such that the free end 536 terminates at the axial midpoint M3 of the inner cell 520 or terminates between the axial midpoint M3 of the inner cell 520 and the inner proximal apex 524 when the frame 500 is in the radially compressed or partially radially expanded configuration. It is also to be appreciated that in some examples, the length L2 of the actuated vertical strut 506 can be shorter than shown in FIG. 9B. Although the actuated struts 506 are described as vertically oriented actuated struts for convenience, it is to be understood that in some examples, the strut may have other nonvertical orientations, such as orientations with a slight angle or curvature relative to an axial axis through the frame.

With continued reference to FIG. 9B, a channel 538 can extend through the second axial member 530 and the actuated vertical strut 506. In some examples, the channel 538 can also extend through the first axial member 528. The channel 538 can be configured to admit at least a portion of the actuator 508, which can extend from the outlet end 518 towards the inlet end 516 of the frame 500. The channel 538 can also be configured to admit at least a portion of a delivery system actuator, such as delivery system actuator 216 shown in FIGS. 5 and 7. In some examples, such as that illustrated in FIG. 9B, the actuated vertical strut 506 can also include a window 540. The window 540 can be configured to accommodate various components of the actuator 408. In some examples, such as those having a rotatably-driven actuator, the window 540 can contain an actuator nut configured to limit the axial range of motion of the actuator 508 as previously discussed.

As shown in FIGS. 9B and 10B, one or more of the vertical struts can include a commissure opening 542. The commissure opening 542 can be disposed between two adjacent outer cells 510, as shown in FIG. 9B, and can be configured to receive one or more leaflet commissures of a valvular structure attached to frame 500. In some examples, such as that illustrated in FIG. 9B, the commissure opening can be closed. Closed commissure openings 542 may advantageously permit a more secure attachment of the leaflets of the valvular structure to the frame 500. However, it is also to be appreciated that the commissure opening 542 may be open, for example at the end of the commissure opening closest to either the inlet end or the outlet end of the frame 500. A commissure opening 542 with an open configuration may, for example, allow the valvular structure to be more easily attached to the frame 500. While FIG. 10 shows the commissure opening 542 formed in a portion of the vertical strut 504 disposed towards the outlet end of the frame 500, it is to be appreciated that in some examples, the commissure opening 542 can be formed a portion of a vertical strut 504 disposed towards the inlet end of the frame 500.

In some examples, the radially inwards extension of the vertical strut 504 having a commissure window 542 shown in FIG. 10B can be accomplished by plastically deforming the vertical strut 504 radially inwards relative to the frame 500, and then applying a shape setting heat treatment to shape set (sometimes called heat set) the vertical strut 504 in this radially deflected configuration. It is to be appreciated, however, that in some examples, the shape setting heat treatment may be omitted, and the plastic deformation of the vertical strut 504 can be sufficient to retain the vertical strut 504 in a configuration in which it extends radially inwards from the rest of the frame.

With continued reference to FIG. 10B, vertical struts 504 having the commissure opening 542 can extend or bow axially inwards from the outer diameter of frame 500. In examples of frame 500 having a vertical strut 504 extending or bowing axially inwards from the outer diameter of the frame, the adjacent angled struts 502 connected to the vertical struts 504 can be deflected inwards to accommodate the vertical strut 504 having the commissure opening 542. In turn, a portion of this radially inwards deflection can be conveyed to the one or more actuated vertical struts 506. In such examples, the radially inwards deflection of the actuated vertical strut 506 resulting from the radially inwards deflection of the vertical strut 504 can minimize the radial displacement of any actuator connected with the frame. For example, the actuated vertical strut 506 can have a reduced radial displacement relative to the inlet end 516 and the outlet end 518 of the frame 500 when the frame 500 is in the compressed configuration. In turn, this can reduce the radial displacement between an end portion 544 and a distal portion 546 of the actuator 508, illustrated in FIG. 9B. This reduced radial displacement can reduce the degree of bending of the actuator 508 when frame 500 is in a radially compressed configuration and can tend to reduce the likelihood of plastic deformation or buckling of the actuator 508.

In some examples, the actuated vertical struts (for example, the actuated vertical strut 130) of a prosthetic heart valve frame (for example, frame 102) can be mechanically supported at both ends. For example, the frame can include an actuated vertical strut attached at a first end to an apex of an inner cell, and attached to other frame components at a second end by one or more lateral and/or angled struts extending from the second end of the actuated vertical strut to other components of the frame. In alternative examples, the second end of the actuated vertical strut can connect directly to a portion of a cell formed by the interconnected struts of the frame. When the prosthetic heart valves according to such examples are held in the radially compressed configuration, the one or more lateral and/or angled struts extending from the second end of the actuated vertical strut, such as actuated vertical strut, can apply a radially compressive force to the second end of the vertical strut. In turn, this radially compressive force can minimize the radial distance between the second end of the actuated vertical strut and the inlet end and outlet end of the frame (such as inlet end 108 and outlet end 110 of the frame 102). As a result, the corresponding bowing of the actuator, (for example, actuator 106) caused by the radial displacement between the end of the actuated vertical strut when the frame is in the radially compressed configuration.

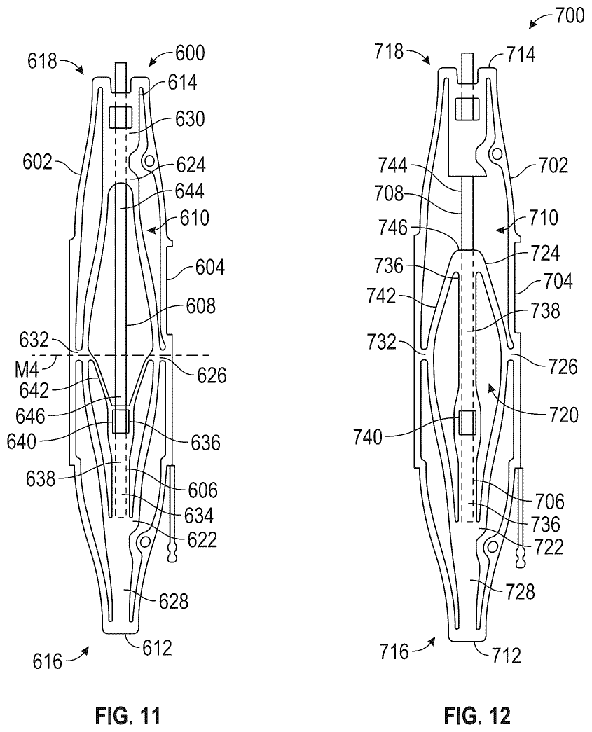

FIG. 11 illustrates one example of a portion of a frame 600 having support for the second end of the actuated strut, in a radially compressed configuration. As shown in FIG. 11, the sections frame 600 can comprise a plurality of angled struts 602, a plurality of vertical struts 604, and one or more actuated vertical struts 606, and can be configured to receive one or more actuators 608. Frame 600 can comprise a plurality of portions similar or substantially identical to the ones shown in FIG. 11 arranged adjacent to each other to form an annular frame for a prosthetic heart valve. In some examples, the frame 600 can comprise six outer cells, but in some examples, a greater or lesser number of outer cells such as 4, 5, 7, 8, 9, 10, 11, or 12 outer cells can make up frame 400.

With continued reference to FIG. 11, the plurality of angled struts 602 and the plurality of vertical struts 604 can form a plurality of outer cells 610 (sometimes called primary cells 610). The outer cells 610 can each have an outer distal apex 612 and an outer proximal apex 614. In some examples, the outer distal apices 612 of the plurality of outer cells 610 can define an inlet end 616 of the frame 600, and he outer proximal apices 614 of the plurality of outer cells 610 can define an outlet end 618 of the frame 600. It is to be understood, however, that in some examples, the outer distal apices 612 may define the outlet end 618 of the frame 600 and the outer proximal apices 614 can define an inlet end 616 of the frame 600. In some examples, each outer cell 610 can be connected in this way to two adjacent outer cells 610 to form an annular frame 600.

As shown in FIG. 11, the angular struts 602 can also form a plurality of inner cells 620 (sometimes called secondary cells 620). The inner cells 620 can each include an inner distal apex 622 and an inner proximal apex 624, as well as two medial vertices 626. Each inner cell 620 can be disposed within a corresponding outer cell 610, as illustrated in FIG. 11. In some examples, such as the one illustrated in FIG. 11, a first axial member 628 can extend from the inner distal apex of the inner cell 620 to the outer distal apex 612 of the corresponding outer cell 610, and a second axial member 630 can extend from the inner proximal apex 624 of the inner cell 620 to the outer proximal apex 614 of the corresponding outer cell 610 to connect the inner cells 620 to the corresponding outer cells 610. In some examples, lateral members 632 can extend from the medial vertices 626 of the inner cells to the vertical struts 604 of the corresponding outer cells. While FIG. 11 shows an inner cell connected to a corresponding outer cell by two axial members and two lateral members, it is to be understood that one or more of these connecting members may, in alternative examples, be absent from the frame 600.

As shown in FIG. 11, the actuated vertical strut 606 can have a first end (sometimes called a fixed end) 634 and a second end (sometimes called a free end) 636. The actuated vertical strut 606 can attach at the first end 634 to the inner distal apex 622 of an inner cell 620, and can extend axially away from the inner distal apex 622 towards the inner proximal apex 624 of the inner cell 620. While FIG. 11 shows that the actuated vertical strut 606 terminates short of the axial midpoint M4 while the frame is in a radially compressed configuration, it is to be appreciated that in alternative examples, the actuated vertical strut 606 can terminate at or past the axial midpoint M4. Although the actuated struts 606 are described as vertically oriented actuated struts for convenience, it is to be understood that in some examples, the strut may have other nonvertical orientations, such as orientations with a slight angle or curvature relative to an axial axis through the frame.

With continued reference to FIG. 11, a channel 638 can extend through the actuated vertical strut 606 and the second axial member 630. The channel 638 can be configured to admit the actuator 608, which can extend from the inlet end 616 end portion of the frame 600 towards the outlet end portion 618 of the frame 600. While FIG. 11 shows an example in which the channel 638 terminates at the inner distal apex 622 of the inner cell 620, it is to be appreciated that in some examples, the channel may extend through the first axial member 628 towards the distal apex 612 of the outer cell. The channel 638 can also be configured to admit components of a delivery system actuator, such as delivery system actuator 216. The actuated vertical strut 606 can also include a window 640. The window 640 can be configured to accommodate various components of the actuator 608. In some examples, such as those having a rotatably-driven actuator, the window 640 can contain an actuator nut configured to limit the axial range of motion of the actuator 608 as previously discussed.

As shown in FIG. 11, the actuated vertical strut 606 can be connected to other components of the frame 600 by one or more lateral support members 642. In examples having an actuated vertical strut 606 that terminates axially short of the inner proximal apex 624 of the free cell (that is, in examples having an actuated vertical strut 606 with a fixed end and a free end), the lateral support members 642 can extend from the second end (that is, free end) 636 of the actuated vertical strut 606 to a component of the frame, such as one or more of the angled struts 604 that define an inner cell 620. In some examples, having an actuated vertical strut 606 that terminates at the inner proximal apex 624 of the inner cell 620, the angled struts 604 that define the inner cell 620 may additionally serve as lateral support members 642. In such examples, the lateral support members 642 may transfer compressive forces from various components of the frame (that is, the angled struts 604 and the actuated vertical struts 606) to the second end 636 of the actuated vertical strut 606. Therefore, when the frame 600 is in a radially compressed state, the lateral support members may tend to apply radially compressive forces on the second end 636 of the actuated vertical strut 606 and thereby cause the actuated vertical strut 606 to more closely conform along its length to the shape of the frame 600 in the compressed configuration. This may reduce the radial displacement between an end portion 644 and a center portion 646 of the actuator 608 caused by the radial compression of the frame 600 and reduce the bending stress on the actuator 608.

In some examples of a section of a frame 700 having support for the free end of the actuated strut, in a radially compressed configuration is shown in FIG. 12. As shown in FIG. 12 the sections frame 700 can comprise a plurality of angled struts 702, a plurality of vertical struts 704, and one or more actuated vertical struts 706, and can be configured to receive one or more actuators 708. Frame 700 can comprise a plurality of portions similar or substantially identical to the ones shown in FIG. 12 arranged adjacent to each other to form an annular frame for a prosthetic heart valve.

As shown in FIG. 12, the plurality of angled struts 702 and the plurality of vertical struts 704 can form a plurality of outer cells 710 (sometimes called primary cells 710). The outer cells 710 can each have an outer distal apex 712 and an outer proximal apex 714. In some examples, the outer distal apices 712 of the plurality of outer cells 710 can define an inlet end 716 of the frame 700, and he outer proximal apices 714 of the plurality of outer cells 710 can define an outlet end 718 of the frame 700. It is to be understood, however, that in some examples, the outer distal apices 712 may define the outlet end 718 of the frame 700 and the outer proximal apices 714 can define an inlet end 716 of the frame 700. In some examples, each outer cell 710 can be connected in this way to two adjacent outer cells 710 to form an annular frame 700. In some examples, the frame 700 can comprise six outer cells, but in some examples, a greater or lesser number of outer cells such as 4, 5, 7, 8, 9, 10, 11, or 12 outer cells can make up frame 400.

In some examples, such as that shown in FIG. 12, the angular struts 702 can also form a plurality of inner cells 720 (sometimes called secondary cells 720). The inner cells 720 can each have an inner distal apex 722 and an inner proximal apex 724, as well as two medial vertices 726. Each inner cell 720 can be disposed within a corresponding outer cell 710 as illustrated in FIG. 12. The inner cell 720 can be connected to the corresponding outer cell by a first axial member 728 extending between the inner distal apex 722 and the outer distal apex 712. In some examples, there may exist no connection between the inner proximal apex 724 and the corresponding outer proximal apex 714, but it is to be understood that in alternative examples, a second axial member may extend between the inner proximal apex 724 and the outer proximal apex 714. In some examples, two lateral members 732 may extend from the medial vertices 726 of the inner cell 720 to the vertical struts 704 of the corresponding outer cell 710.

As shown in FIG. 12, a channel 738 can extend through the actuated vertical strut 706. In some examples, the channel 738 can also extend through the first axial member 728. In examples having a second axial member, the channel 738 can extend through the second axial member. The channel 738 can be configured to admit the actuator 708, which can extend from the inlet end 716 end portion of the frame 700 towards the outlet end portion 718 of the frame 700. While FIG. 11 shows an example in which the channel 738 terminates at the inner distal apex 722 of the inner cell 720, it is to be appreciated that in some examples, the channel may extend further towards the distal apex 712 of the outer cell. The channel 738 can also be configured to admit components of a delivery system actuator, such as delivery system actuator 216. The actuated vertical strut 706 can also include a window 740. The window 740 can be configured to accommodate various components of the actuator 708. In some examples, such as those having a rotatably-driven actuator, the window 740 can contain an actuator nut configured to limit the axial range of motion of the actuator as previously discussed.

With continued reference FIG. 12, the actuated vertical strut 706 can have a first end 734 and a second end 736. The actuated vertical strut 706 can attach at the first end 734 to the inner distal apex 722 of the inner cell 720, and can extend axially away from the inner distal apex 722 and attach to inner proximal apex 724 of the inner cell 720. In this way, the second end 736 of the actuated vertical strut 706 can be coupled to other components of the frame 700, which may apply radially compressive forces on the second end 736 of the actuated vertical strut 706 and thereby cause the actuated vertical strut 706 to more closely conform along its length to the shape of the frame 700 in the compressed configuration. This may reduce the radial displacement between an end portion 744 and a center portion 746 of the actuator 708 caused by the radial compression of the frame 700, and reduce the bending stress on the actuator 708. Although the actuated struts 706 are described as vertically oriented actuated struts for convenience, it is to be understood that in some examples, the strut may have other nonvertical orientations, such as orientations with a slight angle or curvature relative to an axial axis through the frame.

In examples such as those illustrated in FIGS. 11 and 12, the reduced bending stresses on the actuators caused by the radial compression of the frames (that is, frames 600 and 700) can reduce or prevent the buckling of the actuators connected to the frames.

It is to be appreciated that any method of reducing the radial distance (that is, distance R as illustrated in FIG. 4) between an end portion of an actuator and the central portion of the actuator (that is, any method to reduce the bending of the actuator) attached to any of the prosthetic heart valves previously discussed may be used interchangeably. That is, a frame may include an actuated vertical strut with apertures therein, an actuated vertical strut that is heat set or plastically deformed to extend inwards from the outer circumference of the frame, a commissure window heat set or plastically deformed to extend inwards from the outer circumference of the frame, an actuated vertical strut with lateral support members extending therefrom, or any combination thereof.

Also disclosed herein are frames for prosthetic heart valves in which the actuated struts are axial posts (sometimes called vertical posts) with a collapsible window or aperture to facilitate a change in the length of the post along the longitudinal axis of the frame. When stresses are imparted on an axial post including such features, the post can deflect (that is, change shape or elastically deform) to accommodate the actuator and/or to relieve the axial forces acting on the actuator and/or the post. In this way, the degree to which the actuator bends with the frame during the radial compression and/or expansion of the frame can be reduced, and the tendency of the actuator to buckle or bend can be mitigated or prevented.

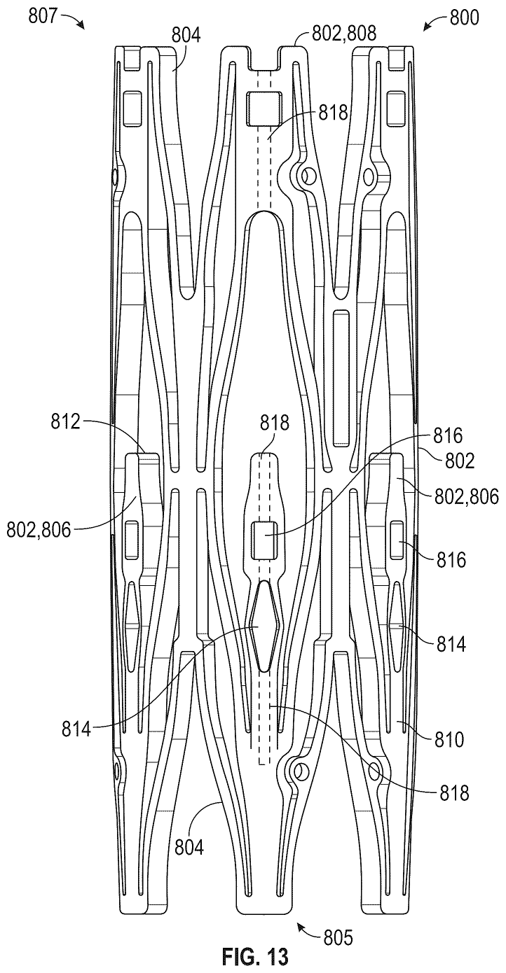

FIG. 13 shows a section of an exemplary prosthetic heart valve frame 800, which includes an axial post with a collapsible window. As shown in FIG. 13, the frame 800 has substantially the same basic configuration as frame 102, as previously described and illustrated in FIGS. 1 and 2, and can generally function in the same way, except for the differences described herein. It is to be understood that, while the frame 800 of FIG. 13 is shown without an attached actuation assembly, actuation assemblies (for example, those previously described in relation to frame 102, and illustrated in FIGS. 1-3) can be used with the frame 800. A prosthetic heart valve can comprise the frame 800 and any of the components described herein for the prosthetic heart valve 100 (for example, leaflets 160, inner and/or outer skirts, connecting members, etc.).

Returning to FIG. 13, the frame 800 comprises a plurality of axially oriented posts 802 and a plurality of interconnected angled struts 804 extending between an inflow end 805 and an outflow end 807. Some of the axially oriented posts 802 are arranged in pairs of first posts 806 and second posts 808, which may be circumferentially aligned and axially spaced apart. The first post 806 can comprise a fixed end portion 810 and a free end portion 812 axially spaced apart and can be cantilevered such that the first post 806 is connected to the other frame components at the fixed end portion 810 and left unconnected to the other frame components at the free end portion 812. The first post 806 can further comprise a collapsible first aperture 814 and, optionally, a second aperture 816 (alternatively called a window 816 or a nut window 816). As shown in FIG. 13, the first aperture 814 can be disposed towards the fixed end portion 810 of the first post 806 and the second aperture 816 can be disposed between the first aperture 814 and the free end portion 812 of the first post 806. While FIG. 13 shows a first post 806 having only the collapsible first aperture 814, it is to be understood that in some examples, there can be more than one collapsible aperture or window in the first post, such as a collapsible second aperture or a collapsible third aperture.

With continued reference to FIG. 13, the first post 806 and the second post 808 can comprise an axially oriented bore 818 (sometimes called a channel) configured to receive an actuator of an actuation assembly. The axially oriented bore 818 can extend through the length of the second post 808, and from the free end portion 812 of the first post 806 towards the inflow end 805 of the frame 800. In some instances, the bore 818 comprises threads. In some such instances, the second aperture 816 and a nut (for example, the nut 146 shown in FIG. 2) can be omitted.

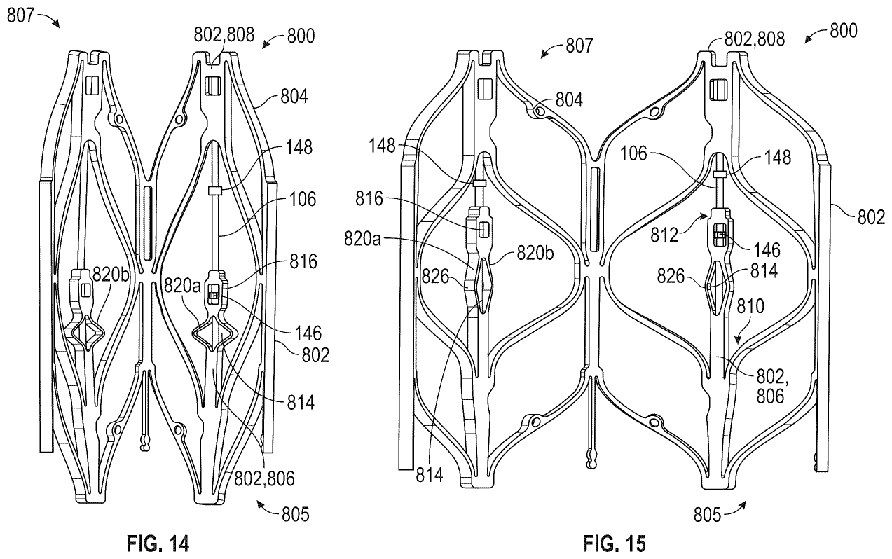

FIGS. 14 and 15 show sections of a frame 800 in the radially compressed and radially expanded state, respectively. As shown in FIGS. 14 and 15, an actuator 106 can extend between the first post 806 and the second post 808. The actuator 106, as discussed herein and illustrated in FIG. 2, can further comprise a lead nut 146 and a stopper 148. The actuator can pass through the bore 818 from the second post 808 to the first post 806. The actuator 106 may also pass through the nut window 816 and the collapsible aperture 814 as illustrated in FIGS. 14 and 15. The lead nut 146 can be disposed in the nut window 816 and can be configured to limit the axial motion of the actuator 106 and to facilitate the radial expansion and compression of the frame 800 as previously discussed. The stopper 148 can be positioned on the actuator 106 between the first post 806 and the second post 808, as shown in FIGS. 14 and 15, and can further limit the axial motion of the actuator 106.

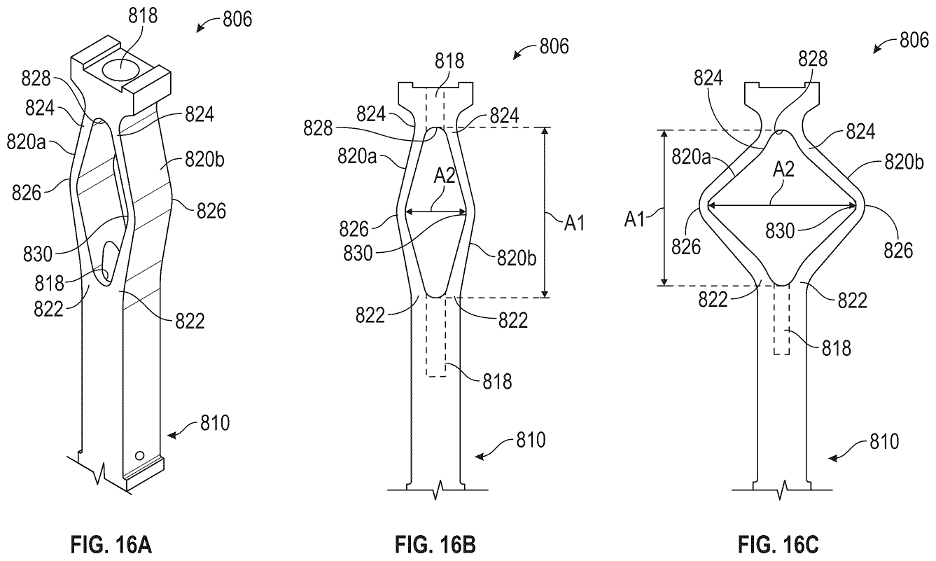

The fixed end portion 810 of the first post 806 with the collapsible aperture 814 is shown in greater detail in FIGS. 16A-16C. As illustrated in FIG. 16A, the collapsible aperture 814 can be defined by a first leg 820a and a second leg 820b, laterally spaced apart from one another. The struts 820a and 820b have a first end portion 822, a second end portion 824, and a joint 826 disposed between the first end portion 822 and the second end portion 824. As illustrated in FIGS. 16A-16C, the first leg 820a and the second leg 820b can be joined at the end portions 822 and 824. In some examples, such as that shown in FIGS. 16A-16C, the joined first leg 820a and second leg 820b form a diamond shaped aperture with a first axis A1 and a second axis A2. The first axis A1 is oriented along the axial length of the first post 806 and extends between two axial vertices 828 formed by the joined end portions 822 and 824 of the legs 820a, 820b. The second axis A2 is oriented transverse to the axial length of the first post 806 and the first axis A1 and extends between two axial vertices 830 located along the joints 826.

As shown in FIG. 16B, the first leg 820a and the second leg 820b can have a variable thickness. In the illustrated example, the first and second struts 820a and 820b can have a greater thickness at the first and second end portions 822, 824, and a lesser thickness at the joint 826. In this way, the first post 806 has a mechanical weak point at the joint 826, which can be used to help ensure that the deflection and/or mechanical deformation of the collapsible aperture 814 occurs primarily at the joints 826.

In some examples, the legs 820a, 820b can have a thickness ranging from 0.15 mm to 0.3 mm at the maximum thickness points along the first and second end portions 822, 824 of each strut. In a specific example, the legs 820a, 820b can have a thickness of 0.2 mm at the maximum thickness points along the first and second end portions 822, 824 of each strut. The legs 820a, 820b can also have a thickness ranging from 0.12 to 0.15 mm at the minimum thickness point at the joint 826 of each strut. In such examples, the legs 820a, 820b can form a collapsible aperture 814 with a height of 3 mm along the first axis A1 and a width of 1.3 mm along the second axis A2, when the window 816 is in an undeflected and/or neutral state (that is, when there are no axially-directed compressive or tensile forces acting on the first post 806).

It will be appreciated by one of ordinary skill in the art that the relative thicknesses of the points of minimum and maximum thickness can be related to one another, and may be selected to control the reaction of the collapsible aperture 814 to axial (that is, tensile or compressive) forces. For example, when the ratio of minimum thickness to maximum thickness is lower, the collapsible aperture 814 may deflect further or under lighter loads, and when the ratio of minimum thickness to maximum thickness is higher, the collapsible aperture 814 can deflect to a lesser degree or require heavier loads to begin deflecting.

In general, when compressive forces act on the first post 806, the legs 820a, 820b forming the collapsible aperture 814 can bend at the joints 826, bringing the axial vertices 828 closer together and pushing the lateral vertices 830 further apart to axially foreshorten the first post 806, as shown in FIG. 16C to an axially compressed state. Conversely, when tensile forces act on the first post 806, or when the compressive force on the first post 806 is removed the legs 820a, 820b forming the collapsible aperture 814 can unbend at the joints 826, bringing the axial vertices 828 further apart and bringing the lateral vertices 830 closer together, as shown in FIG. 16B to axially extend the first post 806 to an axially extended state. Such forces can be caused by the radial expansion and/or contraction of the frame and can be translated to the first post 806 through the actuator 106 as described herein with relation to frames 300, 400, 600, and 700. When the axial length of the first post 806 changes in this way, the shape of the arc that the actuator 106 assumes to accommodate the relative positioning of the first post 806 and the second post 808 can change, and accordingly, the bending stresses on the actuator 106 caused by the change in the curvature of the frame 800 during the deployment of a prosthetic heart valve including the frame 800 can be relieved and/or reduced.

In one example, the prosthetic heart valve including frame 800 can be advanced by a delivery device, such as the delivery device 200 described herein and illustrated in FIGS. 6A through 6C, through the vasculature of the patient to the desired implantation site, and then radially expanded to a desired diameter, as described in greater detail herein.

The prosthetic heart valve including the frame 800 can initially be constrained to a crimped state by a delivery sheath such as the delivery capsule 222 or adjustable loop 224 described herein. While constrained to the crimped state, the frame 800 can have a substantially unbowed shape, and the actuator 106 can be substantially straight along the longitudinal axis of the frame 800. Because the actuator 106 is substantially straight along the longitudinal axis of the frame 800, little or no compressive force is imparted to the first post 806 and the collapsible aperture 814 can remain in the axially extended state.

At the desired implantation site, the prosthetic valve including frame 800 can be deployed from the delivery capsule 222 or the adjustable lasso 224, and the frame 800 can radially expand from the crimped state to a radially compressed state (see FIG. 14). As previously described, the radial expansion can be greater at the axial midsection of the frame 800 than at the inlet end portion 805 or the outlet end portion 807, causing the frame 800 to assume a barreled shape and causing the actuator 106 to bend radially inwards or outwards to accommodate the changing geometry of the frame 800. As the actuator 106 bends radially inwards or outwards, it imparts an axially oriented compressive force to the first post 806. The axially oriented compressive force causes the legs 820a, 820b to bend at the joints 826 to axially collapse the aperture 816 and axially foreshorten the first post 806, as shown in FIGS. 14 and 16C. This in turn can minimize the severity of the bend required to pass the actuator 106 through the bore 818 in both the first post 806 and the second post 808.

The frame 800 can also be mechanically expanded from the radially compressed state to a radially expanded state (see FIG. 15) by rotating the actuator or actuators 106 relative to the first post 806 and the second post 808, in the manner discussed previously in relation to frame 802. As the frame 800 radially expands, diameter of the frame 800 towards the inflow end 805 and the outflow end 807 more closely matches the diameter of the frame 800 towards the axial midpoint of the frame (that is, the frame 800 loses its barreled shape and becomes more cylindrical as it is expanded from the radially compressed state to the radially expanded state). This, in turn, reduces the bending forces on, and thus the severity of the bend of, the actuator 106, allowing the actuator 106 to straighten as the frame 800 radially expands. As the actuator 106 straightens with the radial expansion of the frame 800, the compressive forces imparted to the first post 806 by the actuator 106 are also reduced and the legs 820a, 820b unbend at the joints 826 to axially expand the aperture 816 and axially extend the first post 806, as shown in FIGS. 15 and 16B.

In some circumstances, such as during post ballooning or a valve-in valve procedure, it may also be necessary to expand the diameter of the frame 800 further than can be accomplished solely by the rotation of the actuators 106 relative to the first post 806 and the second post 808. For example, in a valve in valve procedure, a patient has a first prosthetic heart valve pre-installed. In such examples, it may be necessary to replace the valvular structure of the first prosthetic heart valve with the valvular structure of a second prosthetic heart valve. To do this, a second prosthetic heart valve is advanced to the implantation site of the first prosthetic heart valve, and thereafter expanded to the desired diameter. Typically, the desired diameter of the second prosthetic heart valve is large enough to require expansion of the frame of the first prosthetic heart valve to accommodate the second heart valve, such that the frame of the first prosthetic heart valve provides an anchoring site for the second prosthetic heart valve.

In such cases, the frame 800 can be further radially expanded from the radially expanded state to a radially dilated state by applying a force directed radially outwards to the frame 800, which in some examples can be accomplished by inflating an inflatable balloon positioned radially inwards of the frame 800. As the frame 800 is further radially expanded from the radially expanded state to the radially dilated state, the inflow end portion 108 and the outflow end portion 110 are drawn closer together. This in turn causes the ends of the actuator 106 extending between the first post 806 and the second post 808 to draw closer together, compressing and exerting a bending force on the actuator 106. As the actuator 106 is compressed and/or as the actuator 106 begins to bend, it imparts a compressive force on the free end portion 812 of the first post 806, which can cause the legs 820a, 820b to bend at the joints 826 to axially collapse the aperture 816 and axially foreshorten the first post 806 (see FIG. 16C). As described herein, this can relieve the bending forces on the actuator 106 and prevent or mitigate the tendency of the actuator 106 to buckle as the frame 800 is expanded to the radially dilated configuration.

In some examples, the deflection of the legs 820a, 820b (and therefore the deflection of the first post 806) can occur entirely within the elastic region. For example, the legs 820a, 820b may fold along the joints 826 without exceeding the yield stress of the material used. Thus, the components of the first post 806 can, in such examples, experience no plastic deformation during the radial expansion and/or radial compression of the frame. Therefore, when the axial compressive and/or tensile forces imparted to the first post 806 by the actuator 106 are relieved, the legs 820a, 820b and the first post 806 can return to a “neutral” undeflected state. Advantageously, this may allow such an example frame 800 to be radially adjusted multiple times if needed, without plastically deforming one or more components of the first post 806, which may affect further adjustments.

In this way, the compressible aperture 816 can relieve the bending forces experienced by the actuator 106 through the deployment of the prosthetic heart valve including the frame 800. In turn, this mitigates the tendency of the actuator 106 to buckle as the frame is radially expanded and/or compressed to various diameters.

In one specific example, the frame 800 has a diameter of 7 mm while in the radially crimped state within the delivery capsule 222 or the adjustable loop 224, and the first post 806 is in the axially extended state. In this example, when the frame 800 is deployed from the delivery capsule 222 or the adjustable loop 224, the frame 800 expands to a diameter of 13 mm, and the first post 806 deflects from an axially extended state to an axially compressed state. The actuators 106, 316 can then be rotated to expand the frame 800 to a diameter of 27 mm, which causes the first post 806 to deflect from the axially compressed state to the axially extended state. In such an example, the actuators 106, 316 may be unable to radially expand the frame 800 past a diameter of 27 mm, and the frame 800 can be further expanded, for example up to a diameter of 31 mm, by an inflatable balloon, which causes the first post 806 to deflect from the axially extended state to the axially compressed state.

In some examples, the legs 820a, 820b and the compressible aperture 816 formed thereby can be configured to deflect very little while the compressive forces on the first post 806 are below a given threshold, and to deflect greatly while the compressive forces on the first post 806 exceed the given threshold. For example, the collapsible aperture 814 can be configured to function as a mechanical fuse, changing shape as the legs 820a, 820b deflect if the forces on the first post 806 exceed the load threshold.

In this way, the compressible aperture 816 and the first post 806 can be configured to substantially retain their shape while under lower stresses (for example, compressive stresses), such as those which are unlikely to strain the actuator 106 past the yield point and cause plastic deformation, such as that associated with the buckling of the actuators 106, 316. At the same time, the compressible aperture 816 and the first post 806 can also be configured to rapidly deflect under higher stresses to relieve the stresses on the actuator 106 as they approach the yield point and risk plastic deformation and/or catastrophic buckling of the actuator 106.

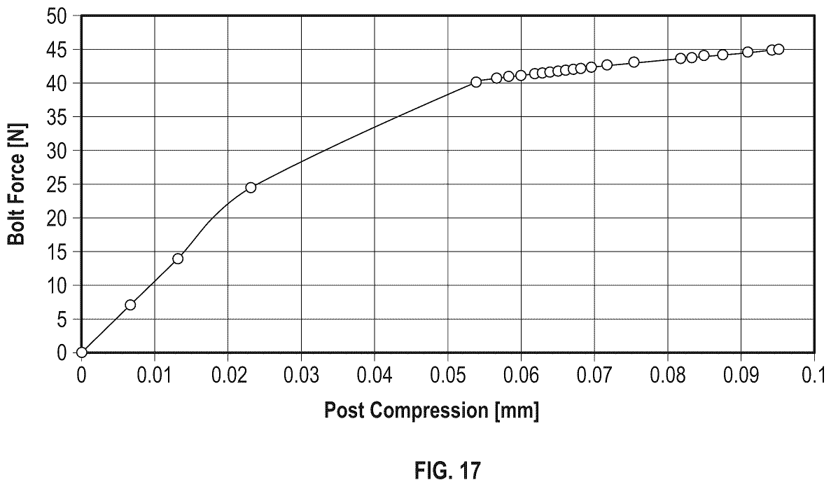

In one particular example, illustrated in FIG. 17, the collapsible aperture 814 is configured to have an actuator force threshold of approximately 40 newtons (40 N). As shown in FIG. 17, in some examples, the collapsible aperture 814 of the first post 806 deflects by less than 0.03 mm when a compressive force of less than or equal to 25 N is applied to the actuator 106 coupled to the frame 800. Similarly, the collapsible aperture 814 of the first post 806 deflects by less than 0.05 mm when a compressive force of less than or equal to 40 N is applied to an actuator 106 coupled to the frame 800. However, the collapsible aperture 814 of the first post 806 deflects by an additional 0.05 mm when a compressive force of 40-45 N is applied to an actuator 106 coupled to the frame 800, for a total deflection approaching 0.1 mm of total deflection at a 45 N compressive force. It should be noted that, for different configurations of the frame 800 and/or the actuator 106 coupled to the frame 800, the actuator force threshold and the corresponding deflection of the collapsible aperture 814 can also be different.

In these ways, the frame 800, having a first post 806 with a collapsible aperture 814 can mitigate or prevent the tendency of the actuator 106 of the prosthetic heart valve assemblies described herein from bending or buckling during the expansion and/or compression of the frame 800, by allowing the first post 806 to relieve the compressive stresses experienced by the actuator 106.

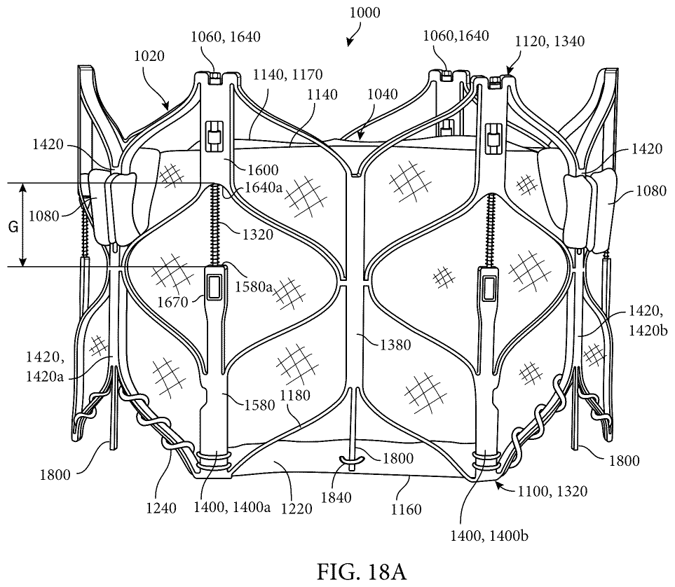

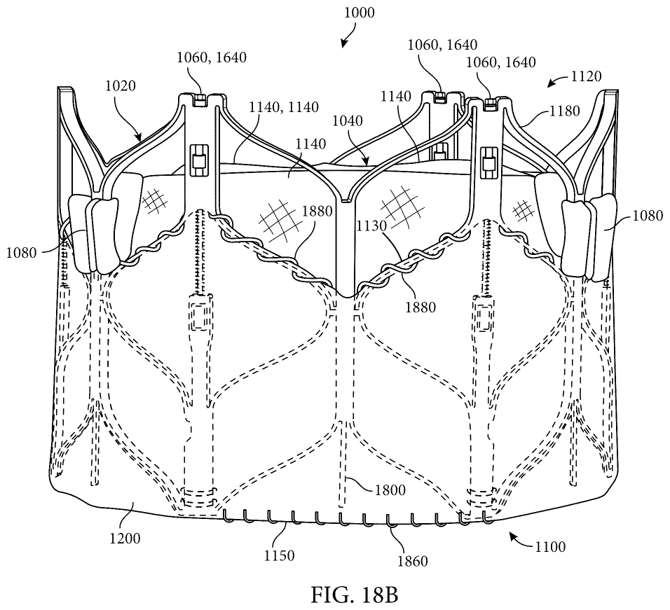

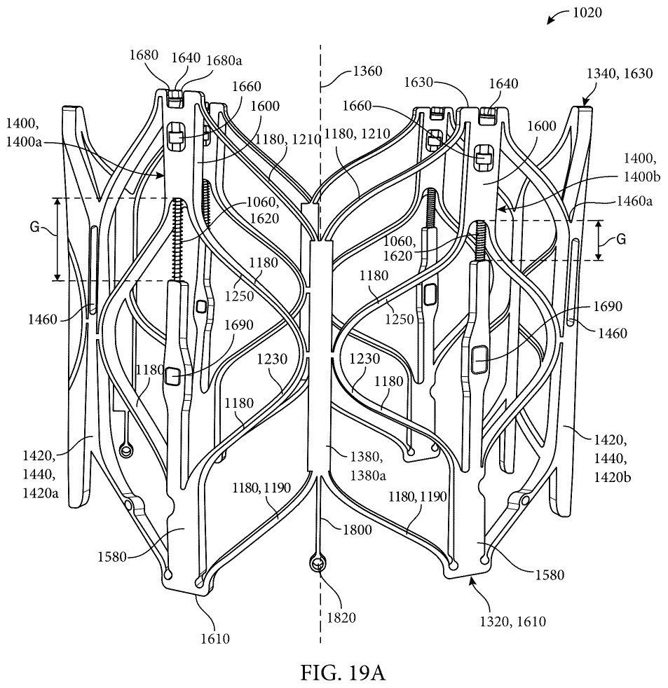

FIGS. 18A and 18B illustrate an exemplary prosthetic valve 1000 (also referred to as prosthetic heart valve), according to one example. The prosthetic valve 1000 includes a frame 1020 having an annular shape. The prosthetic valve 1000 includes a valvular structure 1040 supported inside and coupled to the frame 1020. In some examples, the prosthetic valve 1000 can include rod actuators 1060 that engage actuator portions of the frame 1020 and that can be rotated relative to the actuator portions of the frame 1020 to radially expand and radially compress (or crimp) the frame 1020. In some examples, the rod actuators 1060 and actuator portions of the frame 1020 can cooperate to lock the frame 1020 in a desired radially expanded configuration or radially compressed configuration.

The valvular structure 1040 is configured to regulate the flow of blood through the prosthetic valve 1000, from an inflow end 1100 to an outflow end 1120. The valvular structure 1040 can include, for example, a leaflet assembly comprising one or more leaflets 1140 made of flexible material. The leaflets 1140 can be made in whole or part from, biological material, bio-compatible synthetic materials, or other such materials. Suitable biological material can include, for example, bovine pericardium (or pericardium from other sources). The leaflets 1140 can be secured to one another at their adjacent sides to form commissures 1080, each of which can be secured to portions of the frame 1020.

In the illustrated example of FIGS. 18A and 18B, the valvular structure 1040 includes three leaflets 1140, which can be arranged to collapse in a tricuspid arrangement. Each leaflet 1140 can have an inflow edge portion 1160 (also referred to as cusp edge portion) (FIG. 18A) and an outflow edge portion 1170 (also referred to as free edge portion). The inflow edge portions 1160 of the leaflets 1140 can define an undulating, curved scallop edge that generally follows or tracks portions of struts 1180 of the frame 1020 in a circumferential direction when the frame 1020 is in a radially expanded configuration. The inflow edge portions 1160 of the leaflets 1140 can be referred to as a “scallop line.”

The prosthetic valve 1000 may include one or more skirts mounted around the frame 1020. For example, as shown in FIG. 18B, the prosthetic valve 1000 may include an outer skirt 1200 mounted around an outer surface of the frame 1020. The outer skirt 1200 can function as a sealing member for the prosthetic valve 1000 by sealing against the tissue of the native valve annulus and helping to reduce paravalvular leakage past the prosthetic valve 1000. In some cases, an inner skirt (not shown) may be mounted around an inner surface of the frame 1020. The inner skirt can function as a sealing member to prevent or decrease perivalvular leakage, to anchor the leaflets 1140 to the frame 1020, and/or to protect the leaflets 1140 against damage caused by contact with the frame 1020 during crimping and during working cycles of the prosthetic valve 1000.

In some examples, the inflow edge portions 1160 (shown in FIG. 18A) of the leaflets 1140 can be sutured generally along the scallop line to an inner skirt (not shown) mounted around an inner surface of the frame 1020. The inner skirt can in turn be sutured to adjacent struts 1180 of the frame 1020. In other examples, as shown in FIG. 18A, the leaflets 1140 can be sutured directly to the frame 1020 or to a reinforcing member 1220 (also referred to as a reinforcing skirt or connecting skirt) in the form of a strip of material (for example, a fabric strip) which is then sutured to the frame 1020, along the scallop line via stitches 1240 (for example, whip stitches).

The inner and outer skirts (for example, the outer skirt 1200 shown in FIG. 18B) and the reinforcing member 1220 can be formed from any of various suitable biocompatible materials, which can include any of various synthetic materials, fabrics (for example, polyethylene terephthalate fabric), or natural tissue (for example, pericardial tissue). Further details regarding the use of skirts or sealing members in prosthetic valves can be found, for example, in U.S. Patent Publication No. 2020/0352711, which is incorporated herein by reference.

Further details regarding the assembly of the leaflet assembly and the assembly of the leaflets and the skirts to the frame can be found, for example, in U.S. Provisional Application Nos. 63/209,904, filed Jun. 11, 2021, and 63/224,534, filed Jul. 22, 2021, which are incorporated herein by reference. Further details of the construction and function of the frame 1020 can be found in International Patent Application No. PCT/US2021/052745, filed Sep. 30, 2021, which is incorporated herein by reference.

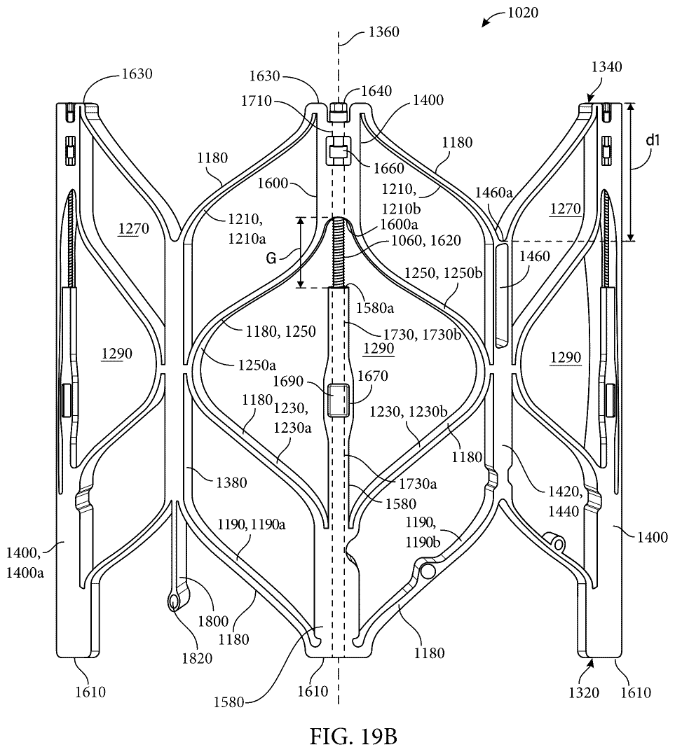

FIGS. 19A and 19B show the frame 1020 with the rod actuators 1060, according to one example. The valvular structure is not shown in FIGS. 19A and 19B. The frame 1020 includes an inflow end 1320 (corresponding to the inflow end 1100 of the prosthetic valve in FIG. 18A), an outflow end 1340 (corresponding to the outflow end 1120 of the prosthetic valve in FIG. 18A), and a longitudinal axis 1360 extending in a direction from the inflow end 1320 to the outflow end 1340. The longitudinal axis 1360 defines the axial direction of the frame 1020.

The frame 1020 includes one or more first frame portions 1400 to which one or more rod actuators 1060 can be coupled. Each first frame portion 1400 can include an inflow actuator portion 1580 (or first post) extending in the axial direction of the frame 1020 and an outflow actuator portion 1600 (or second post) extending in the axial direction of the frame 1020. At the inflow end 1320, an end portion of the inflow actuator portion 1580 forms a first apex 1610 (which can be referred to as “inflow apex”). At the outflow end 1340, an end portion of the second actuator 1600 forms a second apex 1630 (which can be referred to as “outflow apex”). The frame 1020 can include one or more second frame portions 1420 to which the commissures 1080 (shown in FIGS. 18A and 18B) can be coupled. Each second frame portion 1420 can include a commissure support 1440 (or commissure post) extending in the axial direction of the frame 1020.

The frame 1020 can include one or more additional support posts 1380. The first frame portions 1400, second frame portions 1420, and support posts 1380 are spaced about a circumference of the frame 1020 and coupled together by a plurality of circumferentially extending struts 1180. For example, the struts 1180 can extend circumferentially between adjacent frame portions/posts to connect all the frame portions/posts together. In the illustrated example, the struts 1180 have curved shapes, which can allow the struts 1180 to deflect more easily in the axial direction (for example, when radially expanding or radially compressing the frame).

The struts 1180 can include a first row of struts 1190 at or near the inflow end 1320 of the frame 1020, a second row of struts 1210 at or near the outflow end 1340, and third and fourth rows of struts 1230, 1250, respectively, positioned axially between the first and second rows of struts 1190, 1210. As illustrated in FIG. 19B, the struts 1190, 1210, 1230, 1250 (or struts 1180) can form and/or define a plurality of first cells (or openings) 1270 and a plurality of second cells (or openings) 1290 that extend circumferentially around the frame 1020. For example, each first cell 1270 can be formed by struts 1190a, 1190b of the first row of struts 1190, struts 1210a, 1210b of the second row of struts 1210, and posts 1380, 1440. Each second cell 1290 can be formed by struts 1230a, 1230b of the third row of struts 1230 and struts 1250a, 1250b of the fourth row of struts 1250. Each second cell 1290 can be disposed within one of the first cells 1270.

Each first cell 1270 can have an axially-extending hexagonal shape. Each second cell 1290 can have a diamond shape. In some examples, the frame 1020 can comprise six first cells 1270 extending circumferentially in a row, six second cells 1290 extending circumferentially in a row within the six first cells 1270, and twelve support posts 1380. However, in other examples, the frame 1020 can have a greater or fewer number of first cells 1270 and a correspondingly greater or fewer number of second cells 1290 and support posts 1380.

The frame 1020 can have any number, arrangement, and configuration of the frame portions 1400, 1420 and posts 1380. For example, the frame 1020 can have three second frame portions 1420 spaced around the circumference of the frame 1020, which can be used to couple up to three commissures 1080 of a valvular structure to the frame 1020. In one example, the frame 1020 can have six first frame portions 1400, which can be used to couple up to six rod actuators 1060 to the frame 1020. In some examples, the first frame portions 1400 can be grouped in pairs that are spaced around the circumference of the frame 1020 (for example, the first frame portions 1400a, 1400b as indicated in FIG. 19A can form a pair). In some examples, each pair of first frame portions 1400 can be positioned between two spaced-apart second frame portions 1420 (for example, the paired first frame portions 1400a, 1400b between the two spaced-apart second frame portions 1420a, 1420b as indicated in FIG. 19A). In one example, a support post 1380 can be positioned between two first frame portions 1400 (for example, a support post 1380a is shown between the pair of first frame portions 1400a, 1400b in FIG. 19A).

The commissure support 1440 of the second frame portion 1420 can have strut portions defining a commissure window (or commissure opening) 1460. The commissure window 1460 extends radially through a thickness of the commissure support 1440 and can be configured to accept a commissure 1080 (shown in FIG. 18A) of the valvular structure 1040 (shown in FIG. 18A). For example, each commissure 1080 can be mounted to a respective commissure support 1440, such as by inserting a pair of commissure tabs of adjacent leaflets 1140 (shown in in FIG. 18A) through the commissure window 1460 and suturing the commissure tabs to each other and/or the commissure support 1440. In the illustrated example, the commissure window 1460 has a substantially rectangular shape that is shaped and sized to receive commissure tabs of two adjacent leaflets therethrough. In other examples, the commissure window 1460 can have any of various shapes (for example, square, oval, square-oval, triangular, L-shaped, T-shaped, or C-shaped).

The commissure windows 1460 are spaced apart about the circumference of frame 1020. The spacing may or may not be uniform. In one example, the proximal ends 1460a (upper ends in FIG. 19B) of the commissure windows 1460 are axially offset from the outflow end 1340 of the frame 1020 by an offset distance d₁ (shown in FIG. 19B). In some examples, the offset distance d₁ can be in a range from 2 mm to 6 mm. In general, the offset distance d₁ can be selected such that when the leaflets 1140 (shown in FIG. 18A) are attached to the frame 1020 via the commissure windows 1460, the free edge portions 1170 (shown in FIG. 18A) of the leaflets 1140 will not protrude from or past the outflow end 1340 of the frame 1020.

The rod actuator 1060 can include a threaded rod 1620 having a head portion 1640 configured to be releasably coupled to an actuator assembly of a delivery apparatus. The rod actuator 1060 can further include a stopper 1660 (for example, in the form of a nut, washer, collar, or flange) disposed on the threaded rod 1620 such that the stopper 1660 is fixed axially on the threaded rod 1620 and can move in lockstep with the threaded rod 1620 when the threaded rod 1620 is rotated. The stopper 1660 can be integrally formed on the threaded rod or separately formed and coupled to the threaded rod 1620 (for example, by welding, an adhesive, or with a mechanical fastener) such that the stopper 1660 can remain in a fixed axial position on the threaded rod 1620. As will be further described, the stopper 1660 can engage an edge of a stopper window formed in a first frame portion 1400 for radially compressing the frame 1020.

Referring to FIG. 19B, the inflow actuator portion 1580 and the outflow actuator portion 1600 of each first frame portion 1400 are aligned with each other in the axial direction of the frame 1020. A distal end portion of the inflow actuator portion 1580 forms an inflow apex 1610. A proximal end portion of the outflow actuator portion 1600 forms an outflow apex 1630. The outflow actuator portion 1600 includes a distal end 1600a that is in opposing relation to a proximal end 1580a of the inflow actuator portion 1580. The distal end 1580a is separated from the proximal end 1600a by a gap G. The threaded rod 1620 extends through an inner bore 1710 of the outflow actuator portion 1600, across the gap G, and into an inner bore 1730 of the inflow actuator portion 1580.

The inflow actuator portion 1580 can include a nut window 1670 in which a nut 1690 is mounted. The nut 1690 includes a bore with internal threads. The threaded bore of the nut 1690 can be aligned with the inner bore 1730 of the inflow actuator portion 1580 such that the threaded rod 1620 can extend into the inner bore 1730 of the inflow actuator portion 1580 and through the bore of the nut 1690. The threaded rod 1620 includes external threads that engage the internal threads of the bore of the nut 1690. In some examples, the portions of the inner bore 1730 of the inflow actuator portion 1580 located above and below the nut 1690 can have a smooth and/or non-threaded inner surface to allow the threaded rod 1620 to slide freely within the inner bore 1730 while being threadedly engaged with the nut 1690.

In some examples, the nut 1690 can be held in a fixed position relative to the inflow actuator portion 1580 (for example, fixed to the edges of the nut window 1670). Since the nut 1690 is fixed relative to the inflow actuator portion 1580, rotation of the threaded rod 1620 relative to the nut 1690 moves the threaded rod 1620 axially along the nut 1690. When the threaded rod 1620 is not rotated relative to the nut 1690, the threaded engagement between the threaded rod 1620 and the nut 1690 retains the threaded rod 1620 on the inflow actuator portion 1580. Thus, axial movement of the threaded rod 1620 to radially compress or radially expand the prosthetic valve is only possible when the threaded rod 1620 is rotated relative to the nut 1690.

In other examples, in lieu of using the nut 1690, at least a portion of the inner bore 1730 of the inflow actuator portion 1580 can be threaded. For example, the proximal portion 1730b and/or the distal portion 1730a of the inner bore 1730 of the inflow actuator portion 1580 can comprise inner threads that engage the external threads of the threaded rod 1620 such that rotation of the threaded rod 1620 causes the threaded rod 1620 to move axially relative to the inflow actuator portion 1580.

The threaded rod 1620 extending through the inflow and outflow actuator portions 1580, 1600 and threadedly engaged with the inflow actuator portion 1580 can serve as an expansion and locking mechanism for the frame 1020. Such an expansion and locking mechanism can be formed at each of or some of the first frame portions 1400 on the frame 1020. If an expansion and locking mechanism is not desired at a specific first frame portion 1400, a threaded rod 1620 need not extend through the inflow and outflow actuator portions 1580, 1600 of the specific first frame portion. Thus, the frame 1020 can have one or more expansion and locking mechanisms spaced about the circumference of the frame 1020. In the illustrated example, the external threads of the threaded rod 1620 are shown to extend along the rod within the gap G to the distal end 1600a of the outflow actuator portion 1600, however, this is not required. The external threads need only be present on the portion of the threaded rod 1620 that engages the internal threads of the nut 1690 or the internal threads of the inner bore 1730 when the frame is in the radially compressed state and the radially expanded state.

The threaded rod 1620 can be rotated relative to the nut 1690, the inflow actuator portion 1580, and the outflow actuator portion 1600 to axially foreshorten the frame 1020, thereby radially expanding the frame 1020, or to axially elongate the frame 1020, thereby radially compressing the frame 1020. For example, when the threaded rod 1620 is rotated relative to the nut 1690 in one direction, the inflow actuator portion 1580 and the outflow actuator portion 1600 can move axially relative to one another to increase an axial length of the gap G (or widen the gap G), thereby radially compressing the frame 1020 (or the prosthetic valve in general). When the threaded rod 1620 is rotated relative to the nut 1690 in an opposite direction, the inflow actuator portion 1580 and the outflow actuator portion 1600 can move axially relative to one another to decrease the axial length of the gap G (or narrow the gap G), thereby radially expanding the frame 1020 (or the prosthetic valve in general).

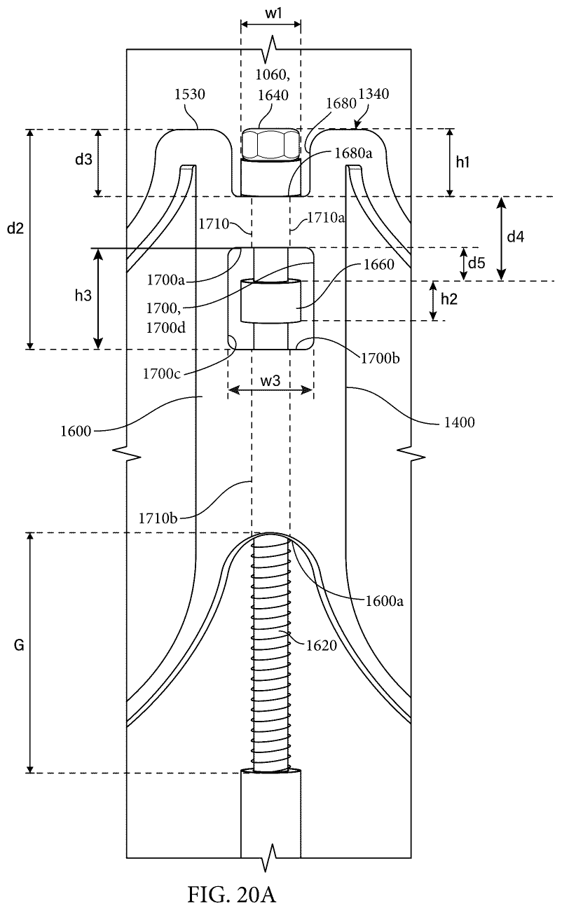

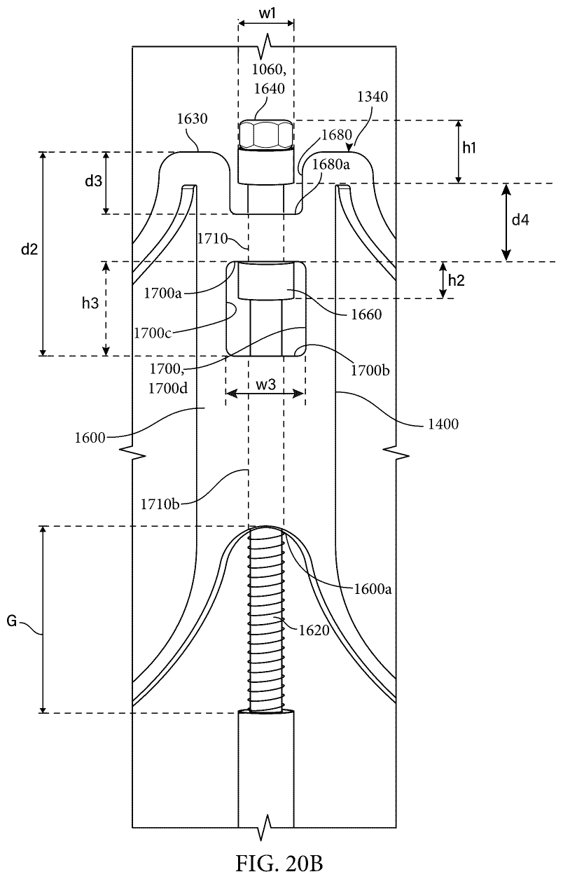

FIG. 20A shows a portion of a first frame portion 1400 including the outflow actuator portion 1600 in greater detail. In the illustrated example, a stopper window 1700 is formed in the outflow actuator portion 1600. The stopper window 1700 extends radially through a thickness of the outflow actuator portion 1600 and intersects the inner bore 1710 of the outflow actuator portion 1600. The stopper window 1700 can have a proximal edge 1700a (which is the upper edge in the figure), a distal edge 1700b (which is the lower edge in the figure), and side edges 1700c, 1700d. The side edges 1700c, 1700d can be parallel to the axial direction of the frame 1020, while the proximal edge 1700a and the distal edge 1700b can be transverse to the axial direction of the frame 1020. The stopper window 1700 is configured to accept the stopper 1660 disposed on the threaded rod 1620 as the threaded rod 1620 extends through the inner bore 1710 of the outflow actuator portion 1600. The stopper 1660 can have a larger diameter compared to the inner bore 1710 so that the stopper 1660 does not move into the portions of the inner bore 1710 above and below the stopper window 1700 and is retained within the stopper window 1700.

In one example, the rod actuator 1060 can be assembled to the first frame portion 1400 by separating the stopper 1660 from the threaded rod 1620, disposing the stopper 1660 within the stopper window 1700 such that the bore of the stopper 1660 is aligned with the inner bore 1710 of the outflow actuator portion 1600, and then inserting the threaded rod 1620 into the inner bore 1710 (for example, from the outflow apex 1630 or outflow end 1340 of the frame) and through the stopper 1660. The stopper 1660 can then be fixedly coupled to the threaded rod 1620 using various techniques and mechanisms, such as by welding, an adhesive, or a mechanical fastener (for example, a pin or screw extending laterally through the stopper and into the threaded rod). Alternatively, other methods of coupling the stopper 1660 to the threaded bore 1620 such that the stopper 1660 is fixed at a desired axial position on the threaded rod 1620 can be used. For example, the stopper 1660 can be a hinged or split collar that can be mounted around the threaded rod 1620 while the threaded rod 1620 extends through the stopper window 1700. The stopper 1660 is not fixed relative to the stopper window 1700 such that the stopper 1660 can move freely within the stopper window 1700, which would allow the stopper 1660 to move in lockstep with the threaded rod 1620 and remain at a fixed axial position on the threaded rod 1620 as the threaded rod 1620 is rotated (for example, during expansion or compression of the frame 1020).

In one example, the frame 1020 can be radially compressed (or crimped) by rotating the rod actuator 1060 in a direction that causes the threaded rod 1620 to move axially in a proximal direction (for example, in a direction towards the outflow end 1340). Since the stopper 1660 is axially fixed in position relative to the threaded rod 1620, the proximal axial movement of the threaded rod 1620 results in proximal axial movement of the stopper 1660 within the stopper window 1700. The stopper 1660 can move proximally (or towards the outflow end 1340) as the threaded rod 1620 is rotated until the stopper 1660 abuts the proximal edge 1700a of the stopper window 1700, as shown in FIG. 20B. Further rotation of the threaded rod 1620 causes the stopper 1660 to apply a proximally directed force to the outflow actuator portion 1600 via contact with the proximal edge 1700a, while the nut 1690 and the inflow actuator portion 1580 move distally along the threaded rod 1620. This causes the inflow actuator portion 1580 to move distally away from the outflow actuator portion 1600, thereby axially lengthening and radially compressing the frame.

Rotating the rod actuator 1060 in the opposite direction causes the head portion 1640 to apply a distally directed force to outflow actuator portion 1600 via contact with an adjacent surface of the frame (for example, surface 1680a), while the nut 1690 and the inflow actuator portion 1580 move proximally along the threaded rod 1620. This causes the inflow actuator portion 1580 to move closer to the outflow actuator portion 1600, thereby axially foreshortening and radially expanding the frame.

The stopper 1660 is fixed axially on the threaded rod 1620 and can be axially offset from the head portion 1640 by a distance d₄ (shown in FIGS. 20A and 20B). The stopper 1660 can have a height h₂ and the stopper window 1700 can have a height h₃ (shown in FIGS. 20A and 20B), wherein h₃ is greater than h₂. The threaded rod 1620 extends through the stopper window 1700 with the head portion 1640 disposed proximally to the stopper window 1700. In some examples, the stopper 1660 can have a lowest axial position within the stopper window 1700 when the head portion 1640 abuts the frame 1020 at the outflow end 1340 (as shown in FIG. 20A) and a highest position within the stopper window 1700 when the stopper 1660 abuts the proximal edge 1700a of the stopper window 1700 (as shown in FIG. 20B). The offset distance d₄ of the stopper 1660 from the head portion 1640, the height h₃ of the stopper window 1700, and the height h₂ of the stopper can be selected such that the stopper 1660 can move axially within the window 1700, from the lowest axial position, toward the proximal edge 1700a of the stopper window 1700. In some cases, the range of axial movement of the stopper 1660 can be selected to allow for over-expansion of the frame. For example, the range of axial movement of the stopper 1660 can be selected such that the stopper 1660 is offset from the proximal edge 1700a by a distance d₅>0 (shown in FIG. 20A) when the frame is radially expanded to a working diameter. The clearance provided by the distance d₅ will allow further expansion of the frame by applying a radial force to the frame, for example, using a balloon or other expansion tool, as further described herein.

The distal edge 1700b of the stopper window 1700 is axially offset from the outflow end 1340 by a distance d₂ (shown in FIGS. 20A and 20B). In one example, the distance d₂ can be selected to be smaller than the distance d₁ (shown in FIG. 19B) by which the proximal edges 1460a of the commissure windows 1460 are axially offset from the outflow end 1340, which positions the stopper window 1700 downstream of the commissure windows 1460 (or at axial positions between the commissure windows 1460 and the outflow end 1340). Positioning the stopper window 1700 downstream of the commissure windows 1460 or relatively closer to the outflow end 1340 compared to the commissure windows 1460 can advantageously distance the stopper 1660 in the stopper window 1700 from soft components (such as skirts and/or leaflets) of the prosthetic valve.

The leaflets 1140 (shown in FIG. 18A) can be pressed against the frame 1020 when the prosthetic valve is in the radially compressed state. In some examples, the dimensions (for example, height h₃ and width w₃ as shown in FIGS. 20A and 20B) of the stopper window 1700 are selected to be large enough to freely accommodate the stopper 1660 (that is, allow the stopper 1660 to rotate with the threaded rod 1620 and move axially with the threaded rod 1620 within the stopper window 1700) and small enough to prevent portions of the leaflets 1140 from penetrating into the stopper window 1700 when the frame is radially compressed.

In some examples, as illustrated in FIGS. 20A and 20B, a recess 1680 can be formed in the end portion of the second actuator portion 1600 including an outflow apex 1630. The recess 1680 can be configured to receive the head portion 1640 of the rod actuator 1060. The recess 1680 can be connected to the inner bore 1710 of the outflow actuator portion 1600 such that the threaded rod 1620 can be inserted into the inner bore 1710 through the recess 1680. In one example, the width w₁ (or diameter) of the head portion 1640 is greater than the diameter of a proximal portion 1710a of the inner bore 1710 such that the head portion 1640 does not move into the inner bore 1710 as the threaded rod 1620 extends through the inner bore 1710, which would position the head portion 1640 proximally to the stopper window 1700 as previously described.

In one example, a depth d₃ of the recess 1680 can be equal to or greater than a height h₁ of the head portion 1640 such that the head portion 1640 can be fully disposed (or hidden) within the recess 1680 (that is, the proximal end of the head portion 1640 is flush or recessed relative to the outflow end 1340 as illustrated, for example, in FIG. 20A) at least when the frame 1020 is radially expanded to a working diameter. Hiding the head portion 1640 within the recess 1680 while the frame 1020 is radially expanded to a working diameter can advantageously reduce the risk of interactions between the head portion 1640 and the surrounding anatomy when the prosthetic valve 1000 is implanted within the anatomy. While the frame 1020 is in a radially compressed configuration or not radially expanded to the working diameter, depending on the depth d₃ of the recess 1680 relative to the height h₁ of the head portion 1640, the threaded rod 1620 may extend into the recess 1680 or even past the recess 1680 such that the head portion 1640 protrudes from the outflow end 1340 (as illustrated, for example, in FIG. 20B).

To radially expand the prosthetic valve, the threaded rod 1620 can be rotated relative to the nut 1690 (shown in FIGS. 19A and 19B) until the head portion 1640 engages a distal edge (or bottom edge) 1680a of the recess 1680. While the head portion 1640 is engaged with the distal edge 1680a, the head portion 1640 can prevent the outflow actuator portion 1600 from moving proximally relative to the threaded rod 1620 and can apply a distally directed force to the outflow actuator portion 1600. While the head portion 1640 is engaged with the distal edge 1680a, the threaded rod 1620 can be rotated relative to the nut 1690 in a direction that extends the threaded rod 1620 farther into the inflow actuator portion 1580. Since the head portion 1640 is engaged with the distal edge 1680a, the inflow actuator portion 1580 will be drawn closer to the head portion 1640 as the threaded rod 1620 extends farther into the inflow actuator portion 1580. The gap G between the inflow actuator portion 1580 and the outflow actuator portion 1600 narrows as the inflow actuator portion 1580 draws closer to the head portion 1640, causing the frame 1020 to be axially foreshortened and radially expanded.

In some examples, after radially expanding the frame 1020 (or prosthetic valve) to a working diameter, the stopper 1660 is at an axial position within the stopper window 1700 that is offset from the proximal edge 1700a of the stopper window 1700 by a distance d₅>0 (as illustrated in FIG. 20A). In some examples, this offset distance d₅ allows the radially-expanded frame 1020 to be over-expanded to a diameter greater than the working diameter. For example, an expansion tool, such as an inflatable balloon, can be inserted within the frame 1020 and operated to apply an outward radial force to the frame 1020 that expands the frame. The distance d₅ allows the outflow actuator portion 1600 to move axially towards the inflow actuator portion 1580 in response to the applied radial force, which results in further narrowing of the gap G and further axial foreshortening of the frame 1020 to an over-expanded diameter.

As the outflow actuator portion 1600 moves towards the inflow actuator portion 1580, the proximal edge 1700a of the stopper window moves towards the stopper 1660. The frame 1020 can be over-expanded via the expansion tool until the proximal edge 1700a of the stopper window 1700 engages the stopper 1660, at which point the distance d₅ is now zero and over-expansion of the frame is no longer possible. Therefore, the stopper 1660 also acts to limit over-expansion of the frame 1020. One practical use of over-expanding the frame 1020 is to allow reshaping or remodeling of the annulus into a more circular shape, thereby allowing the prosthetic valve to be deployed uniformly in a circular shape. When the outward radial force applied by the expansion tool is removed (for example, the inflatable balloon is deflated), the frame 1020 returns to its initial expanded state, and the annulus can relax and form a seal around the prosthetic valve.

The inflow end portion of one or more of the posts 1380, 1440 can include an extension 1800 (shown in FIGS. 18A, 19A, and 19B) that extends towards the inflow end 1320 of the frame 1020. In some examples, the extension 1800 can include an aperture 1820 extending radially through a thickness of the extension 1800 (as shown in FIGS. 19A and 19B). In some examples, the extension 1800 can extend such that an inflow edge of the extension 1800 aligns with or substantially aligns with the inflow end 1320 of the frame 1020. In use, the extension 1800 can prevent or mitigate portions of an outer skirt from extending radially inwardly and thereby prevent or mitigate any obstruction of flow through the frame 1020 caused by the outer skirt. The extensions 1800 can further serve as supports to which portions of the inner and/or outer skirts and/or the leaflets and/or the connecting skirt 1220 can be coupled (as shown in FIG. 18A). For example, sutures used to connect the inner and/or outer skirts and/or the leaflets and/or the connecting skirt 1220 can be wrapped around the extensions 1800 and/or can extend through apertures 1820. In a particular example, the inflow edge portion 1160 of each leaflet 1140 can be connected to a corresponding extension 1800 via a suture 1840 (as shown in FIG. 18A).

In some examples, the outer skirt 1200 can be mounted around the outer surface of frame 1020 as shown in FIG. 18B, and the inflow edge 1150 of the outer skirt 1200 (lower edge in FIG. 18B) can be attached to the connecting skirt 1220 and/or the inflow edge portions 1160 of the leaflets 1140 that have already been secured to the frame 1020 as well as to the extensions 1800 of the frame by sutures 1860. The outflow edge 1130 of the outer skirt 1200 (the upper edge in FIG. 18B) can be attached to selected struts with stitches 1880. In implementations where the prosthetic valve includes an inner skirt, the inflow edge of the inner skirt can be secured to the inflow edge portions 1160 of the leaflets 1140 before securing the inflow edge portions 1160 to the frame 1020 so that the inner skirt will be between the leaflets 1140 and the inner surface of the frame 1020. After the inner skirt and leaflets 1140 are secured in place, then the outer skirt 1200 can be mounted around the frame 1020 as described above.

The frame 1020 can be a unitary and/or fastener-free frame that can be constructed from a single piece of material (e.g., Nitinol, stainless steel or a cobalt-chromium alloy), such as in the form of a tube. The plurality of cells 1270, 1290 (shown in FIG. 19B) can be formed by removing portions (e.g., via laser cutting) of the single piece of material. The threaded rods 1620 can be separately formed and then inserted through the inner bores in the struts 1580, 1600 of the actuator posts 1400 and threaded through the nuts 1690 of the first posts 1600 of the rod actuator 1400.

In some examples, the frame 1020 can be formed from a plastically-expandable material, such as stainless steel or a cobalt-chromium alloy. When the frame is formed from a plastically-expandable material, the prosthetic valve 1000 can be placed in a radially compressed state along the distal end portion of a delivery apparatus for insertion into a patient's body. When at the desired implantation site, the frame 1020 (and therefore the prosthetic valve 1000) can be radially expanded from the radially compressed state to a radially expanded state via actuation of actuation assemblies of the delivery apparatus (as further described below), which rotate the threaded rods 1620 to produce expansion of the frame 1020. During delivery to the implantation site, the prosthetic valve 1000 can be placed inside of a delivery capsule (sheath) to protect against the prosthetic valve contacting the patient's vasculature, such as when the prosthetic valve is advanced through a femoral artery. The capsule can also retain the prosthetic valve in a compressed state having a slightly smaller diameter and crimp profile than may be otherwise possible without a capsule by preventing any recoil (expansion) of the frame once it is crimped onto the delivery apparatus.

In other examples, the frame 1020 can be formed from a self-expandable material (e.g., Nitinol). When the frame 1020 is formed from a self-expandable material, the prosthetic valve can be radially compressed and placed inside the capsule of the delivery apparatus to maintain the prosthetic valve in the radially compressed state while it is being delivered to the implantation site. When at the desired implantation site, the prosthetic valve is deployed or released from the capsule. In some examples, the frame (and therefore the prosthetic valve) can partially self-expand from the radially compressed state to a partially radially expanded state. The frame 1020 (and therefore the prosthetic valve 1000) can be further radially expanded from the partially expanded state to a further radially expanded state via actuation of actuation assemblies of the delivery apparatus (as further described below), which rotate the rods 1620 to produce expansion of the frame.

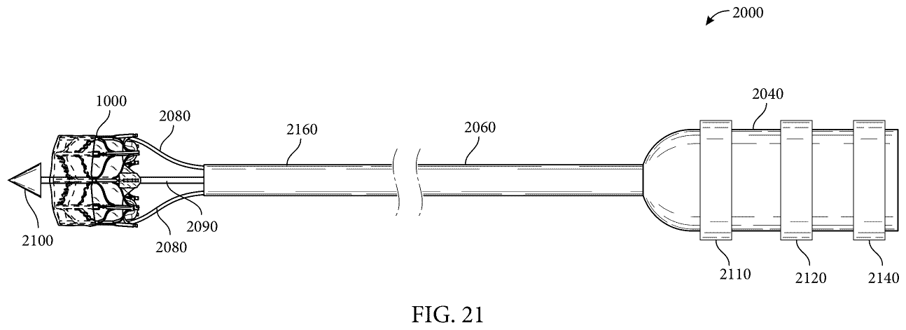

FIG. 21 illustrates an exemplary delivery apparatus 2000 that can be used to deliver the prosthetic valve 1000 to a desired implantation location. The delivery apparatus 2000 and other delivery apparatuses disclosed herein can be used to implant prosthetic devices other than prosthetic valves, such as stents or grafts. The prosthetic valve 1000 can be releasably coupled to the delivery apparatus 2000 via the rod actuators 1060.

In the illustrated example, the delivery apparatus 2000 generally includes a handle 2040, a first elongated shaft 2060 (which comprises an outer shaft) extending distally from the handle 2040, one or more actuator assemblies 2080 extending distally through the first shaft 2060, a second elongated shaft 2090 (which comprises an inner shaft) extending through the first shaft 2060, and a nosecone 2100 coupled to a distal end portion of the second shaft 2090. The second shaft 2090 and the nosecone 2100 can define a guidewire lumen for advancing the delivery apparatus through a patient's vasculature over a guidewire. Each actuator assembly 2080 can be configured to radially expand and/or radially collapse the prosthetic valve 1000 when actuated, such as by one or more knobs 2110, 2120, 2140 of the handle 2040.

Though the illustrated example shows two actuator assemblies 2080 for purposes of illustration, it should be understood that one actuator assembly 2080 can be provided for each actuator (for example, rod actuator 1060/threaded rod 1620) on the prosthetic valve. For example, three actuator assemblies 2080 can be provided for a prosthetic valve having three actuators. In other embodiments, a greater or fewer number of actuator assemblies can be present.

In some examples, a distal end portion 2160 of the shaft 2060 can be sized to house the prosthetic valve in its radially compressed, delivery state during delivery of the prosthetic valve through the patient's vasculature. In this manner, the distal end portion 2160 functions as a delivery sheath or capsule for the prosthetic valve during delivery.

The actuator assemblies 2080 can be releasably coupled to the prosthetic valve 1000. In the illustrated example, each actuator assembly 2080 can be coupled to a respective actuator (for example, rod actuator 1060/threaded rod 1620 in FIGS. 18A-20B) of the prosthetic valve 1000. Each actuator assembly 2080 can include a support tube and an actuator member. When actuated, the actuator assembly can transmit pushing and/or pulling forces to portions of the prosthetic valve to radially expand and collapse the prosthetic valve as previously described. The actuator assemblies 2080 can be at least partially disposed radially within, and extend axially through, one or more lumens of the first shaft 2060. For example, the actuator assemblies 2080 can extend through a central lumen of the shaft 2060 or through separate respective lumens formed in the shaft 2060.

The handle 2040 of the delivery apparatus 2000 can include one or more control mechanisms (for example, knobs or other actuating mechanisms) for controlling different components of the delivery apparatus 2000 in order to expand and/or deploy the prosthetic valve 1000. In the illustrated example, the handle 2040 includes first, second, and third knobs 2110, 2120, and 2140, respectively.

The first knob 2110 can be a rotatable knob configured to produce axial movement of the first shaft 2060 relative to the prosthetic valve 1000 in the distal and/or proximal directions in order to deploy the prosthetic valve from the delivery sheath 2160 once the prosthetic valve has been advanced to a location at or adjacent the desired implantation location with the patient's body. For example, rotation of the first knob 2110 in a first direction (e.g., clockwise) can retract the sheath 2160 proximally relative to the prosthetic valve 1000 and rotation of the first knob 2110 in a second direction (e.g., counter-clockwise) can advance the sheath 2160 distally. In other embodiments, the first knob 2110 can be actuated by sliding or moving the first knob 2110 axially, such as pulling and/or pushing the knob. In other embodiments, actuation of the first knob 2110 (rotation or sliding movement of the first knob 2110) can produce axial movement of the actuator assemblies 2080 (and therefore the prosthetic valve 1000) relative to the delivery sheath 2160 to advance the prosthetic valve distally from the sheath 2160.

The second knob 2120 can be a rotatable knob configured to produce radial expansion and/or compression of the prosthetic valve 1000. For example, rotation of the second knob 2120 can rotate the threaded rods of the prosthetic valve 1000 via the actuator assemblies 2080. Rotation of the second knob 2120 in a first direction (e.g., clockwise) can radially expand the prosthetic valve 1000 and rotation of the second knob 2120 in a second direction (e.g., counter-clockwise) can radially collapse the prosthetic valve 1000. In other embodiments, the second knob 2120 can be actuated by sliding or moving the second knob 2120 axially, such as pulling and/or pushing the knob.

The third knob 2140 can be a rotatable knob operatively connected to a proximal end portion of each actuator assembly 2080. The third knob 2140 can be configured to retract an outer sleeve or support tube of each actuator assembly 2080 to disconnect the actuator assemblies 2080 from the proximal portions of the actuators of the prosthetic valve (e.g., threaded rod). Once the actuator assemblies 2080 are uncoupled from the prosthetic valve 1000, the delivery apparatus 2000 can be removed from the patient, leaving just the prosthetic valve 1000 in the patient.

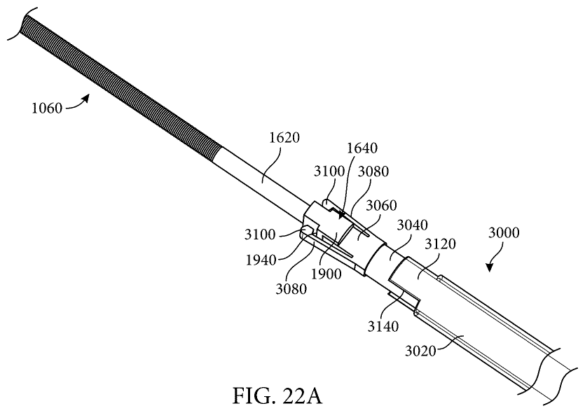

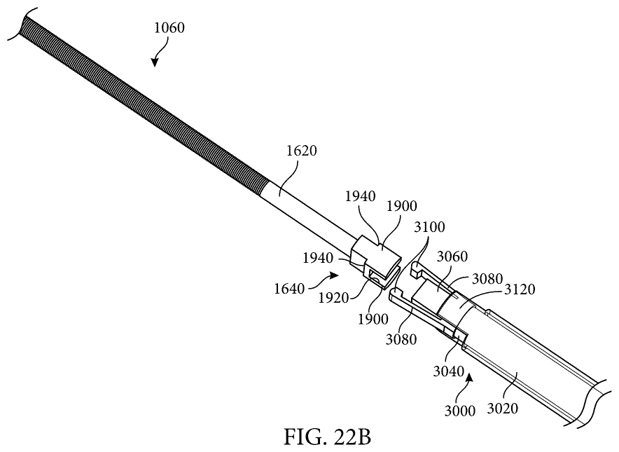

FIGS. 22A and 22B illustrate how each of the threaded rods 1620 of the prosthetic valve 1000 can be removably coupled to an exemplary actuator assembly 3000 of a delivery apparatus (for example, an actuator assembly 2080 of the delivery apparatus 2000). Specifically, FIG. 22A illustrates how a threaded rod 1620 can be coupled to an actuator assembly 3000, while FIG. 22B illustrates how the threaded rod 1620 can be detached from the actuator assembly 3000.

In some examples, the head portion 1640 of the threaded rod 1620 is configured to be releasably coupled to a respective actuator assembly 3000 of the delivery apparatus 2000. For example, the head portion 1640 can include first and second protrusions 1900, defining a channel or slot 1920 between them, and one or more shoulders 1940.

Each actuator assembly 3000 can include a first actuation member configured as a support tube or outer sleeve 3020 and a second actuation member configured as a driver 3040. The driver 3040 can extend through the outer sleeve 3020 (the outer sleeve 3020 is shown transparently in FIGS. 22A and 22B for purposes of illustration). The distal end portions of the outer sleeve 3020 and driver 3040 can be configured to engage or abut a proximal end of the threaded rod 1620 (for example, the head portion 1640) and/or a proximal end of the frame 1020 (for example, an outflow apex 1630 shown in FIGS. 19A and 19B).

The proximal portions of the outer sleeve 3020 and driver 3040 can be operatively coupled to the handle of a delivery apparatus (for example, the handle 2040 shown in FIG. 21). The delivery apparatus in this example can include the same features described previously for delivery apparatus 2000. In particular examples, the proximal end portions of each driver 3040 can be operatively connected to the knob 2120 (shown in FIG. 21) such that rotation of the knob 2120 (clockwise or counterclockwise) causes corresponding rotation of the drivers 3040. The proximal end portions of each outer sleeve 3020 can be operatively connected to the knob 2140 (shown in FIG. 21) such that rotation of the knob 2140 (clockwise or counterclockwise) causes corresponding axial movement of the sleeves 3020 (proximally or distally) relative to the drivers 3040. In other examples, the handle can include electric motors for actuating these components.

The distal end portion of the driver 3040 can include a central protrusion 3060 configured to extend into the slot 1920 of the head portion 1640 of the rod actuator 1060. The distal end portion of the driver 3040 can include one or more flexible elongated elements or arms 3080 having protrusions or teeth 3100 configured to be releasably coupled to the shoulders 1940 of the head portion 1640 of the rod actuator 1060. The protrusions 3100 can extend radially inwardly toward a longitudinal axis of the second actuation member 3040. As shown in FIGS. 22A and 22B, the elongated elements 3080 can be configured to be biased radially outward to an expanded state, for example, by shape setting the elements 3080.

As shown in FIG. 22A, to couple the actuator assembly 3000 to the threaded rod 1620, the driver 3040 can be positioned such that the central protrusion 3060 is disposed within the slot 1920 of the head portion 1640 and such that the protrusions 3100 of the elongated elements 3080 are positioned distally to the shoulders 1940 of the head portion 1640. As the outer sleeve 3020 is advanced (for example, distally) over the driver 3040, the sleeve 3020 compresses the elongated elements 3080 they abut and/or snap over the shoulders 1940, thereby coupling the actuator assembly 3000 to the rod actuator 1060. Thus, the outer sleeve 3020 effectively squeezes and locks the elongated elements 3080 and the protrusions 3100 of the driver 3040 into engagement with (that is, over) the shoulders 1940 of the head portion 1640, thereby coupling the driver 3040 to the rod actuator 1060.

Because the central protrusion 3060 of the driver 3040 extends into the slot 1920 of the head portion 1640 when the driver 3040 and the rod actuator 1060 are coupled, the driver 3040 and the rod actuator 1060 can be rotationally locked such that they co-rotate. So coupled, the driver 3040 can be rotated (for example, using knob 2120 of the handle of the delivery apparatus 2000 shown in FIG. 21) to cause corresponding rotation of the rod actuator 1060 to radially expand or radially compress the prosthetic device. The central protrusion 3060 can be configured (for example, sized and shaped) such that it is advantageously spaced apart from the inner walls of the outer sleeve 3020, such that the central protrusion 3060 does not frictionally contact the outer sleeve 3020 during rotation. Though in the illustrated example the central protrusion 3060 has a substantially rectangular shape in cross-section, in other embodiments, the protrusion 3060 can have any of various shapes, for example, square, triangular, oval, etc. The slot 1920 can be correspondingly shaped to receive the protrusion 3060.

The outer sleeve 3020 can be advanced distally relative to the driver 3040 past the elongated elements 3080, until the outer sleeve 3020 engages the frame 1020 (for example, an outflow actuator portion 1600 of the frame 1020). The distal end portion of the outer sleeve 3020 also can comprise first and second support extensions 3120 defining gaps or notches 3140 between the extensions 3120. The support extensions 3120 can be oriented such that, when the actuator assembly 3000 is coupled to a respective rod actuator 1060, the support extensions 3120 extend partially over an adjacent end portion (for example, the upper end portion) of a respective outflow actuator portion 1600 on opposite sides of the outflow actuator portion 1600. The engagement of the support extensions 3120 with the frame 1020 in this manner can counteract rotational forces applied to the frame 1020 by the rod actuator 1060 during expansion of the frame 1020. In the absence of a counter-force acting against these rotational forces, the frame can tend to “jerk” or rock in the direction of rotation of the rods when they are actuated to expand the frame. The illustrated configuration is advantageous in that outer sleeves, when engaging the second posts 1600 of the frame 1020, can prevent or mitigate such jerking or rocking motion of the frame 1020 when the frame 1020 is radially expanded.

To decouple the actuator assembly 3000 from the prosthetic device 1000, the sleeve 3020 can be withdrawn proximally relative to the driver 3040 until the sleeve 3020 no longer covers the elongated elements 3080 of the driver 3040. As described above, the sleeve 3020 can be used to hold the elongated elements 3080 against the shoulders 1940 of the head portion 1640 of the rod actuator 1060 since the elongated elements 3080 can be naturally biased to a radial outward position where the elongated elements 3080 do not engage the shoulders 1940 of the head portion 1640. Thus, when the sleeve 3020 is withdrawn such that it no longer covers/constrains the elongated elements 3080, the elongated elements 3080 can naturally and/or passively deflect away from, and thereby release from, the shoulders 1940 of the threaded rod 1620, thereby decoupling the driver 3040 from the rod actuator 1060. The sleeve 3020 can be advanced (moved distally) and/or retracted (moved proximally) relative to the driver 3040 via a control mechanism (for example, knob 2140 shown in FIG. 21) on the handle 2040 of the delivery apparatus 2000, by an electric motor, and/or by another suitable actuation mechanism. For example, the physician can turn the knob 2140 in a first direction to apply a distally directed force to the sleeve 3020 and can turn the knob 2140 in an opposite second direction to apply a proximally directed force to the sleeve 3020. Thus, when the sleeve 3020 does not abut the prosthetic device and the physician rotates the knob 2140 in the first direction, the sleeve 3020 can move distally relative to the driver 3040, thereby advancing the sleeve 3020 over the driver 3040. When the sleeve 3020 does abut the prosthetic device, the physician can rotate the knob 2140 in the first direction to push the entire prosthetic device distally via the sleeve 3020. Further, when the physician rotates the knob 2140 in the second direction the sleeve 3020 can move proximally relative to the driver 3040, thereby withdrawing/retracting the sleeve 3020 from the driver 3040.

### Delivery Techniques

For implanting the prosthetic valve within an anatomy, the prosthetic valve can be delivered to the implantation site in a radially compressed configuration. At the delivery site, the prosthetic valve can be radially expanded to the working diameter. When the frame of the prosthetic valve is expanded to the working diameter, the head portion 1640 of the rod actuator 1060 can be recessed relative to the outflow end of the frame (for example, hidden within a recess formed at an outflow apex 1630 of the frame as illustrated in FIG. 20A).

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (for example, by inflating a balloon, actuating one or more actuators of the delivery apparatus, or deploying the prosthetic valve from a delivery capsule to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire and/or an introducer sheath previously inserted into a patient's vasculature. Moreover, the disclosed delivery approaches are not intended to be limited. Any of the prosthetic valves disclosed herein can be implanted using any of various delivery procedures and delivery devices known in the art.

Advantageously, prosthetic heart valves according to the examples previously discussed can reduce the bending stresses on the attached actuators by reducing the radial displacement between the end portions and the center portions of the actuators. In turn, this may mitigate the tendency of the actuators to bend or buckle during the implantation procedure and reduce resulting impairment to the ability of the prosthetic heart valve to be radially expanded or contracted at the desired implantation site.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples heat/thermal sterilization include steam sterilization and autoclaving. Examples of radiation for use in sterilization include, without limitation, gamma radiation, ultra-violet radiation and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Additional Examples of the Disclosed Technology

Example 1: A prosthetic valve comprises a frame having an axial direction extending from an inflow end to an outflow end, a first frame portion extending in the axial direction, and a first window formed in the first frame portion. The prosthetic valve comprises a threaded rod coupled to the first frame portion and extending through the first window, wherein rotation of the threaded rod relative to the first frame portion in a first direction radially expands the frame and rotation of the threaded rod relative to the first frame portion in a second direction that is opposite to the first direction radially compresses the frame. The prosthetic valve comprises a stopper disposed within the first window and coupled to the threaded rod, wherein the stopper is fixed in axial position relative to the threaded rod and axially movable within the first window by rotation of the threaded rod, and wherein the stopper can engage a first edge of the window to produce radial compression of the frame.

Example 2: The prosthetic valve of any example herein, particularly Example 210, wherein the first frame portion comprises a first actuator portion and a second actuator portion aligned in the axial direction, wherein an end portion of the first actuator portion forms a first apex at the inflow end, wherein an end portion of the second actuator portion forms a second apex at the outflow end, and wherein the first window is formed in the second actuator portion.

Example 3: The prosthetic valve of any example herein, particularly Example 2, wherein the first actuator portion includes a first end, wherein the second actuator portion includes a second end in opposing relation to the first end and separated from the first end by a gap, and wherein rotation of the threaded rod in the first direction decreases a size of the gap and rotation of the threaded rod in the second direction increases the size of the gap.

Example 4: The prosthetic valve of any example herein, particularly Example 3, wherein the first window is connected to the first end by a first bore formed in the second actuator portion, and wherein a diameter of the stopper is larger than a diameter of the first bore to prevent passage of the stopper through the first bore.

Example 5: The prosthetic valve of any example herein, particularly Example 4, wherein the threaded rod extends through the stopper.

Example 6: The prosthetic valve of any example herein, particularly any one of Examples 4-5, wherein the threaded rod includes a head portion, and further comprising a recess formed in the second apex to receive the head portion.

Example 7: The prosthetic valve of any example herein, particularly Example 6, wherein a depth of the recess is equal to or greater than a height of the head portion.

Example 8: The prosthetic valve of any example herein, particularly any one of Examples 6-7, wherein the recess is connected to the first window by a second bore formed in the second actuator portion, and wherein a diameter of the stopper is larger than a diameter of the second bore to prevent passage of the stopper through the second bore.

Example 9: The prosthetic valve of any example herein, particularly Example 8, wherein the threaded rod extends through the second bore, the first window, and the first bore, and wherein a diameter of the head portion is larger than a diameter of the second bore to prevent passage of the head portion through the second bore.

Example 10: The prosthetic valve of any example herein, particularly Example 2-9, wherein the threaded rod is threadedly engaged with the first actuator portion, and wherein rotation of the threaded rod relative to the first actuator portion radially expands or radially compresses the frame.

Example 11: The prosthetic valve of any example herein, particularly Example 10, wherein the first actuator portion includes a threaded bore or a threaded nut that threadedly engages with the threaded rod.

Example 12: The prosthetic valve of any example herein, particularly any one of Examples 2-11, further comprising a valvular structure disposed inside the frame and having at least one commissure.

Example 13: The prosthetic valve of any example herein, particularly Example 12, wherein the frame comprises a second frame portion spaced from the first frame portion along a circumference of the frame, and wherein the second frame portion comprises a second window configured to receive the at least one commissure.

Example 14: The prosthetic valve of any example herein, particularly Example 13, wherein the first window is positioned between the outflow end and the second window in the axial direction.

Example 15: The prosthetic valve of any example herein, particularly any one of Examples 13-14, wherein the frame comprises a plurality of first struts connecting the first actuator portion to the second frame portion and a plurality of second struts connecting the second actuator portion to the second frame portion.

Example 16: The prosthetic valve of any example herein, particularly any one of Examples 1-15, further comprising a skirt assembly coupled to the frame.

Example 17: The prosthetic valve of any example herein, particularly any one of Examples 1-16, further comprises a plurality of first frame portions extending in the axial direction and spaced along a circumference of the frame and a plurality of first windows formed in the plurality of first frame portions. The prosthetic valve further comprises a plurality of threaded rods coupled to the plurality of first frame portions and extending through the plurality of first windows, wherein rotation of each threaded rod relative to the respective first frame portion in a first direction radially expands the frame and rotation of each threaded rod relative to the respective first frame portion in a second direction that is opposite to the first direction radially compresses the frame. The prosthetic valve further comprises a plurality of stoppers disposed within the plurality of first windows and coupled to the plurality of threaded rods extending through the plurality of first windows, wherein each stopper is fixed in axial position relative to the respective threaded rod and axially movable within the respective first window by rotation of the respective threaded rod, and wherein each stopper can engage a first edge of the respective first window to produce radial compression of the frame.

Example 18: A prosthetic valve comprises a frame having an axial direction extending from an inflow end to an outflow end, a first frame portion extending in the axial direction, a second frame portion extending in the axial direction, a first window formed in the first frame portion, a second window formed in the second frame portion, wherein the first window is positioned axially between the second window and the outflow end in the axial direction. The prosthetic valve comprises a threaded rod coupled to the first frame portion and extending through the first window, wherein rotation of the threaded rod relative to the first frame portion in a first direction radially expands the frame and rotation of the threaded rod relative to the first frame portion in a second direction that is opposite to the first direction radially compresses the frame. The prosthetic valve comprises a stopper fixedly coupled to the threaded rod and disposed within the first window, wherein the stopper can engage a first edge of the first window to produce radial compression of the frame. The prosthetic valve comprises a valvular structure disposed inside the frame and having a commissure received in the second window.

Example 19: The prosthetic valve of any example herein, particularly Example 18, wherein the first frame portion includes a first actuator portion and a second actuator portion aligned in the axial direction, wherein an end portion of the first actuator portion forms a first apex at the inflow end, wherein an end portion of the second actuator portion forms a second apex at the outflow end, and wherein the first window is formed in the second actuator portion.

Example 20: The prosthetic valve of any example herein, particularly Example 19, wherein the first actuator portion has a first end, wherein the second actuator portion has a second end in opposing relation to the first end and separated from the first end by a gap, and wherein the rod actuator extends across the gap.

Example 21: The prosthetic valve of any example herein, particularly any one of Examples 19-20, wherein the threaded rod extends through the stopper.

Example 22: The prosthetic valve of any example herein, particularly any one of Examples 19-21, wherein a recess is formed in the second apex, and wherein the threaded rod includes a head portion received in the recess.

Example 23: The prosthetic valve of any example herein, particularly Example 22, wherein a depth of the recess is equal to or greater than a height of the head portion.

Example 24: The prosthetic valve of any example herein, particularly any one of Examples 19-23, wherein the frame comprises a plurality of first struts connecting the first actuator portion to the second frame portion and a plurality of second struts connecting the second actuator portion to the second frame portion.

Example 25: The prosthetic valve of any example herein, particularly any one of Examples 18-24, further comprising a skirt assembly coupled to the frame.

Example 26: The prosthetic valve of any example herein, particularly any one of Examples 18-25, further comprises a plurality of first frame portions extending in the axial direction and spaced along a circumference of the frame and a plurality of first windows formed in the plurality of first frame portions. The prosthetic valve comprises a plurality of threaded rods coupled to the plurality of first frame portions and extending through the plurality of first windows, wherein rotation of each of the plurality of threaded rods relative to the respective first frame portion in the first direction radially expands the frame and rotation of each of the plurality of threaded rods relative to the respective first frame portion in a second direction that is opposite to the first direction radially compresses the frame. The prosthetic valve comprises a plurality of stoppers fixedly coupled to the plurality of threaded rods and disposed within the plurality of first windows, wherein each of the plurality of stoppers can engage a first edge of the respective first window to produce radial compression of the frame.

Example 27: A prosthetic valve comprises a frame having an axial direction extending from an inflow end to an outflow end, a first frame portion extending in the axial direction, the first frame portion comprising a first actuator portion connected to the inflow end at a first apex, a second actuator portion connected to the outflow end at a second apex, and a recess formed in the second apex. The prosthetic valve comprises a threaded rod including a head portion, the threaded rod extending through the first actuator portion and the second actuator portion and threadedly engaged with the first actuator portion with the head portion received in the recess, wherein a height of the head portion is equal to or less than a depth of the recess, wherein rotation of the threaded rod relative to the first actuator portion in a first direction radially expands the frame and rotation of the threaded rod relative to the first actuator portion in a second direction that is opposite to the first direction radially compresses the frame.

Example 28: The prosthetic valve of any example herein, particularly Example 27, further comprising a first window formed in the second actuator portion and axially displaced from the recess, wherein the threaded rod extends through the first window.

Example 29: The prosthetic valve of any example herein, particularly Example 28, further comprising a stopper fixedly coupled to the threaded rod and disposed within the first window, wherein the stopper can engage a first edge of the first window to produce radial compression of the frame.

Example 30: The prosthetic valve of any example herein, particularly any one of Examples 28-29, further comprising a valvular structure disposed inside the frame and having at least one commissure.

Example 31: The prosthetic valve of any example herein, particularly Example 30, wherein the frame comprises a second frame portion spaced from the first frame portion along a circumference of the frame, and wherein the second frame portion comprises a second window configured to receive the at least one commissure.

Example 32: The prosthetic valve of any example herein, particularly Example 31, wherein the first window is positioned axially between the outflow end and the second window in the axial direction.

Example 33: The prosthetic valve of any example herein, particularly any one of Examples 27-32, further comprises a plurality of first frame portions extending in the axial direction and spaced along a circumference of the frame, the plurality of first frame portions comprising a plurality of first actuator portions connected to the inflow end at a plurality of first apices, a plurality of second actuator portions connected to the output end at a plurality of second apices, and a plurality of recesses formed in the plurality of second apices. The prosthetic valve comprises a plurality of threaded rods extending through the plurality of first actuator portions and the plurality of second actuator portions and threadedly engaged with the plurality of first actuator portions, each of the plurality of threaded rods having a head portion received in a respective recess, wherein rotation of each of the plurality of threaded rods relative to the respective first actuator portion in a first direction radially expands the frame and rotation of each of the plurality of threaded rods relative to the first action actuator in a second direction radially compresses the frame.

Example 34: A prosthetic valve comprises a frame having an axial direction extending from an inflow end to an outflow end, a plurality of first frame portions spaced about a circumference of the frame, and a first window formed in each of the first frame portions. The prosthetic valve comprises a plurality of threaded rods, each threaded rod coupled to one of the first frame portions and extending through the respective first window formed in the one of the first frame portions, wherein rotation of each threaded rod relative to the respective first frame portion in a first direction radially expands the frame and rotation of each threaded rod relative to the respective first frame portion in a second direction that is opposite to the first direction radially compresses the frame. The prosthetic valve comprises at least one stopper fixedly coupled to one of the threaded rods and disposed within the first window receiving the one of the threaded rods, wherein the at least one stopper can engage a first edge of the respective first window to produce radial compression of the frame.

Example 35: The prosthetic valve of any example herein, particularly Example 34, wherein a stopper is fixedly coupled to each of the threaded rods and disposed within the first window receiving the threaded rod.

Example 36: The prosthetic valve of any example herein, particularly any one of Examples 34-35, wherein each first frame portion comprises a first actuator portion and a second actuator portion aligned in the axial direction, wherein the first actuator portion is connected to the inflow end, wherein the second actuator portion is connected to the outflow end, and wherein the first window of the first frame portion is formed in the second actuator portion.

Example 37: The prosthetic valve of any example herein, particularly Example 36, wherein the first actuator portion of each first frame portion has a first end, wherein the second actuator portion of each first frame portion has a second end in opposing relation to the first end and separated from the first end by a gap, and wherein the rod actuator coupled to each first frame portion extends across the respective gap.

Example 38: The prosthetic valve of any example herein, particularly Example 36, wherein the threaded rod extends through the at least one stopper.

Example 39: The prosthetic valve of any example herein, particularly Example 38, wherein the at least one stopper comprises a threaded bore, and wherein the threaded rod is threadedly engaged with the threaded bore.

Example 40: The prosthetic valve of any example herein, particularly any one of

Examples 38-39, further comprising a recess formed in an end portion of the second actuator portion adjacent to the outflow end, and wherein the threaded rod includes a head portion disposed in the recess.

Example 41: The prosthetic valve of any example herein, particularly Example 40, wherein a depth of the recess is equal to or greater than a height of the head portion.

Example 42: The prosthetic valve of any example herein, particularly any one of Examples 34-41, wherein the frame comprises a plurality of second frame portions circumferentially spaced about the circumference of the frame and a second window formed in each of the second frame portions.

Example 43: The prosthetic valve of any example herein, particularly Example 42, wherein the first windows are positioned closer to the outflow end compared to the second windows in the axial direction.

Example 44: The prosthetic valve of any example herein, particularly Example 43, further comprising a valvular structure disposed inside the frame, the valvular structure having at least one commissure received in one of the second windows.

Example 45: The prosthetic valve of any example herein, particularly any one of Examples 34-44, comprising a plurality of stoppers, wherein each stopper is coupled to one of the plurality of threaded rods and disposed within a respective first window receiving the threaded rod, and wherein each stopper can engage a first edge of the respective first window to produce radial compression of the frame.

Example 46: The prosthetic valve of any example herein, particularly any one of Examples 1-45, wherein the prosthetic valve is sterilized.

Example 47: A method comprises coupling a prosthetic valve to a distal end of a delivery apparatus, wherein the prosthetic valve comprises a frame having an axial direction extending from an inflow end to an outflow end and having a first actuator portion and a second actuator portion aligned along the axial direction, a threaded rod coupled to the first actuator portion and extending axially through the second actuator portion, and a stopper fixedly coupled to the threaded rod and disposed within a window formed in the second actuator portion. The method comprises rotating the threaded rod in a first direction to cause the stopper to engage a first edge of the window, thereby causing the first actuator portion to move away from the second actuator portion and radially compress the prosthetic valve.

Example 48: The method of any example herein, particularly Example 47, wherein rotating the threaded rod in the first direction axially displaces the stopper in a direction towards the outflow end.

Example 49: The method of any example herein, particularly any one of Examples 47-48, further comprising rotating the threaded rod in a second direction that is opposite to the first direction to radially expand the prosthetic valve to a first diameter and disengage the stopper from the first edge of the window.

Example 50: The method of any example herein, particularly Example 49, wherein the threaded rod is rotated until a head portion of the threaded rod is fully retracted into a recess formed in an end portion of the second actuator portion connected to the outflow end.

Example 51: The method of any example herein, particularly Example 49, further comprising applying an outward radial force to the frame to over-expand the prosthetic valve to a second diameter that is greater than the first diameter and removing the outward radial force from the frame, wherein the prosthetic valve returns to the first diameter after removing the outward radial force.

Example 52: The method of any example herein, particularly Example 51, wherein applying the outward radial force to the frame displaces the first edge of the window in a direction towards the stopper, and wherein engagement of the first edge of the window with the stopper during applying the outward radial force limits over-expansion of the prosthetic valve.

Example 53: The method of any example herein, particularly any one of Examples 47-52, wherein the frame comprises a plurality of first actuator portions aligned with a plurality of second actuator portions, wherein a plurality of threaded rods are coupled to the first actuator portions and extend axially through the respective second actuator portions, wherein a plurality of stoppers are fixedly coupled to the plurality of threaded rods and disposed in respective windows formed in the plurality of second actuator portions, and wherein each of the plurality of threaded rods is rotated in a first direction to cause the respective stopper to engage an edge of the respective window.

Example 54: The method of any example herein, particularly any one of Examples 47-53, further comprises inserting the prosthetic valve and the distal end of the delivery apparatus into a patient's vasculature and advancing the delivery apparatus through the patient's vasculature to position the prosthetic valve at an implantation site.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. A prosthetic valve comprising:
a frame having an axial direction extending from an inflow end to an outflow end, a first frame portion extending in the axial direction, and a first window formed in the first frame portion;
a threaded rod coupled to the first frame portion and extending through the first window, wherein rotation of the threaded rod relative to the first frame portion in a first direction radially expands the frame and rotation of the threaded rod relative to the first frame portion in a second direction that is opposite to the first direction radially compresses the frame; and
a stopper disposed within the first window and coupled to the threaded rod, wherein the stopper is fixed in axial position relative to the threaded rod and axially movable within the first window by rotation of the threaded rod, and wherein the stopper can engage a first edge of the window to produce radial compression of the frame.

2. The prosthetic valve of claim 1, wherein the first frame portion comprises a first actuator portion and a second actuator portion aligned in the axial direction, wherein an end portion of the first actuator portion forms a first apex at the inflow end, wherein an end portion of the second actuator portion forms a second apex at the outflow end, and wherein the first window is formed in the second actuator portion.

3. The prosthetic valve of claim 2, wherein the first actuator portion includes a first end, wherein the second actuator portion includes a second end in opposing relation to the first end and separated from the first end by a gap, and wherein rotation of the threaded rod in the first direction decreases a size of the gap and rotation of the threaded rod in the second direction increases the size of the gap.

4. The prosthetic valve of claim 3, wherein the first window is connected to the first end by a first bore formed in the second actuator portion, and wherein a diameter of the stopper is larger than a diameter of the first bore to prevent passage of the stopper through the first bore.

5. The prosthetic valve of claim 4, wherein the threaded rod extends through the stopper.

6. The prosthetic valve of claim 4, wherein the threaded rod includes a head portion, and further comprising a recess formed in the second apex to receive the head portion.

7. The prosthetic valve of claim 6, wherein a depth of the recess is equal to or greater than a height of the head portion.

8. The prosthetic valve of claim 6, wherein the recess is connected to the first window by a second bore formed in the second actuator portion, and wherein a diameter of the stopper is larger than a diameter of the second bore to prevent passage of the stopper through the second bore.

9. The prosthetic valve of claim 8, wherein the threaded rod extends through the second bore, the first window, and the first bore, and wherein a diameter of the head portion is larger than a diameter of the second bore to prevent passage of the head portion through the second bore.

10. The prosthetic valve of claim 2, wherein the threaded rod is threadedly engaged with the first actuator portion, and wherein rotation of the threaded rod relative to the first actuator portion radially expands or radially compresses the frame.

11. The prosthetic valve of claim 10, wherein the first actuator portion includes a threaded bore or a threaded nut that threadedly engages with the threaded rod.

12. The prosthetic valve of claim 2, further comprising a valvular structure disposed inside the frame and having at least one commissure.

13. The prosthetic valve of claim 12, wherein the frame comprises a second frame portion spaced from the first frame portion along a circumference of the frame, and wherein the second frame portion comprises a second window configured to receive the at least one commissure.

14. The prosthetic valve of claim 13, wherein the first window is positioned between the outflow end and the second window in the axial direction.

15. The prosthetic valve of claim 13, wherein the frame comprises a plurality of first struts connecting the first actuator portion to the second frame portion and a plurality of second struts connecting the second actuator portion to the second frame portion.

16. The prosthetic valve of claim 1, further comprising a skirt assembly coupled to the frame.

17. The prosthetic valve of claim 1, further comprising:
a plurality of first frame portions extending in the axial direction and spaced along a circumference of the frame and a plurality of first windows formed in the plurality of first frame portions;
a plurality of threaded rods coupled to the plurality of first frame portions and extending through the plurality of first windows, wherein rotation of each threaded rod relative to the respective first frame portion in a first direction radially expands the frame and rotation of each threaded rod relative to the respective first frame portion in a second direction that is opposite to the first direction radially compresses the frame; and
a plurality of stoppers disposed within the plurality of first windows and coupled to the plurality of threaded rods extending through the plurality of first windows, wherein each stopper is fixed in axial position relative to the respective threaded rod and axially movable within the respective first window by rotation of the respective threaded rod, and wherein each stopper can engage a first edge of the respective first window to produce radial compression of the frame.
